SEQUENCE LISTING (15 sequences)

SEQ ID NO: 1 - DNA, 27 bp, Artificial Sequence
  synthetic construct; forward primer
aagatagtcctatcaaatcatggaacg

SEQ ID NO: 2 - DNA, 27 bp, Artificial Sequence
  synthetic construct; reverse primer
catagcgttggctacccgtgatattgc

SEQ ID NO: 3 - DNA, 27 bp, Artificial Sequence
  synthetic construct; reverse primer
gccgctacaggacagataagagacgaa

SEQ ID NO: 4 - DNA, 20 bp, Artificial Sequence
  synthetic construct; human PKG2 forward primer
agcccgcttcaggcctctcc

SEQ ID NO: 5 - DNA, 20 bp, Artificial Sequence
  synthetic construct; human PKG2 reverse primer
agtcgaccctctgccagcac

SEQ ID NO: 6 - DNA, 30 bp, Artificial Sequence
  synthetic construct; human Sox9 forward primer
gatctgaagaaggagagcgaggaggacaag

SEQ ID NO: 7 - DNA, 25 bp, Artificial Sequence
  synthetic construct; human Sox9 reverse primer
cgttgggggagatgtgcgtctgctc

SEQ ID NO: 8 - DNA, 20 bp, Artificial Sequence
  synthetic construct; human MUC2 forward primer
ccgctggagccgtatctgcg

SEQ ID NO: 9 - DNA, 20 bp, Artificial Sequence
  synthetic construct; human MUC2 reverse primer
cggggcagggcaggtctttg

SEQ ID NO: 10 - DNA, 20 bp, Artificial Sequence
  synthetic construct; human CDX2 forward primer
aggcagaagagccgcgagga

SEQ ID NO: 11 - DNA, 20 bp, Artificial Sequence
  synthetic construct; human CDX2 reverse primer
ccagtcctcccggagtgggg

SEQ ID NO: 12 - DNA, 20 bp, Artificial Sequence
  synthetic construct; mouse PKG2 forward primer
gtggcggaggacgccaagac

SEQ ID NO: 13 - DNA, 20 bp, Artificial Sequence
  synthetic construct; mouse PKG2 reverse primer
aggcctgcagtgggcttcct

SEQ ID NO: 14 - DNA, 24 bp, Artificial Sequence
  synthetic construct; HPRT1 forward primer
gcgtcgtgattagcgatgatgaac

SEQ ID NO: 15 - DNA, 24 bp, Artificial Sequence
  synthetic construct; HPRT1 reverse primer
cctcccatctccttcatgacatct

(19) United States
(12) Patent Application Publication
(10) Pub. No.: US 2013/0123264 A1
(43) Pub. Date: May 16, 2013

(54) COMPOSITIONS AND METHODS FOR TREATMENT OF INFLAMMATORY BOWEL DISORDERS AND INTESTINAL CANCERS

(71) Applicant: Darren D. Browning, Augusta, GA (US)

(72) Inventor: Darren D. Browning, Augusta, GA (US)

(21) Appl. No.: 13/654,118

(22) Filed: Oct. 17, 2012

Related U.S. Application Data
(60) Provisional application No. 61/565,142, filed on Nov. 30, 2011.
(60) Provisional application No. 61/548,127, filed on Oct. 17, 2011.

(51) Int. Cl. A61K 31/53; A61K 31/519

(52) U.S. Cl. CPC A61K 31/53; A61K 31/519
USPC 514/243; 514/252.16

(57) ABSTRACT

Compositions and formulations useful for treating inflammatory bowel diseases, intestinal cancers, and improving the integrity of the luminal surface of the gastrointestinal tract are disclosed. Typically, the composition increases level of cGMP in the intestinal mucosa or increases the activity of a PKG, preferably PKG2, in the intestinal mucosa. Preferably, the composition includes a phosphodiesterase type 5 (PDE5) inhibitor and is targeted to the intestinal mucosa, for example by enteric coating or attachment of an intestinal specific targeting moiety.

Methods of using the compositions to treat intestinal and bowel diseases, and intestinal cancers, and methods of improving one or more physiological parameters of intestinal luminal integrity including, but are not limited to, reduced proliferation for example in the intestinal crypts, reduced apoptosis for example at the luminal surface, and induction of downstream effectors of PKG-mediated signaling are also disclosed.

### CROSS REFERENCE TO RELATED APPLICATIONS

This application claims priority to and benefit of U.S. Provisional Application No. 61/548,127 filed Oct. 17, 2011 and U.S. Provisional Application No. 61/565,142 filed Nov. 30, 2011, and where permissible are incorporated herein in their entirety.

### FIELD OF THE INVENTION

The field of the invention generally relates to compositions and methods for treating intestinal diseases and disorders such as intestinal cancers and inflammatory bowel diseases.

### BACKGROUND OF THE INVENTION

Continuous renewal of the luminal epithelium is essential for maintaining a healthy intestine and disruption of this process is thought to be a major cause of gastrointestinal disease. The balance between cellular proliferation at the crypt base and terminal differentiation at the luminal surface is referred to as intestinal homeostasis and is highly regulated (Scoville D H, et al., Gastroenterology, 134:949-64 (2008)). Numerous signaling pathways have been identified as important organizers of the crypt-villus axis. One of the most widely studied is Wnt signaling through β-catenin/T-cell factor (TCF), which has a critical role in the maintenance of intestinal stem cells at the crypt base (Simons, et al., Experimental Cell Research (2011)). The stem cells give rise to proliferative progenitor cells that undergo cell fate decisions in the transit amplifying region of the crypt. The commitment of these cells to either secretory (goblet and enteroendocrine cells) or enterocyte lineage is controlled in part by Notch and bone morphogenetic protein pathways (Medema, et al., Nature, 474:318-326 (2011)).

Uroguanylin and guanylin are endogenous peptide hormones that regulate solute and water balance in the intestine (Forte L R, Jr. Pharmacol Ther, 104:137-42 (2004)). These ligands bind to guanylyl cyclase C receptors (GCC) expressed on the surface of intestinal epithelial cells (IEC) and trigger intracellular cGMP production (Steinbrecher, et al, Current Opinion in Gastroenterology, 27:139-145 (2011)). Effectors for cGMP include phosphodiesterases, ion channels, and cGMP-dependent protein kinases (PKG) (Hofmann, et al, Handb Exp Pharmacol, 137-62 (2009) and Hofmann, et al., Physiol Rev, 86:1-23 (206)). There are two genes encoding mammalian cGMP-dependent protein kinases, with alternate splicing of the type 1 gene to produce 3 isoforms. Type 1 alpha and beta isoforms are nearly ubiquitous and soluble, whereas type 2 PKG (PKG2) is thought to be more tissue restricted (brain, intestine, kidney) and has an amino-terminal lipid anchor.

Type 1 PKG is widely distributed but is best-characterized as a regulator of smooth muscle relaxation (Lincoln, et al., J. Appl Physiol, 91:1421-30 (2001)). Type 2 PKG is more tissue restricted, but in IEC it mediates C1-secretion in response to guanylin/uroguanylin by phosphorylating cystic fibrosis transmembrane conductance regulator (Markert, et al., J. Clin Invest, 96:822-30 (1995) and Pfeifer, et al., Science, 274:2082-6 (1996)). PKG activation is traditionally by cGMP derived from NO-stimulated soluble guanylyl cyclase or from membrane/receptor associated particulate guanylyl cyclases. The cGMP signal is terminated by the activity of phosphodiesterases which cleave the 3′-5′ phosphoester linkage to generate GMP.

In addition to controlling electrolyte balance, the guanylin/GCC/cGMP pathway has emerged as an important regulator of intestinal homeostasis. Guanylin knockout mice exhibit reduced cGMP levels in the colonic mucosa and have an increased proliferative compartment (Steinbrecher, et al., Am J Pathol, 161:2169-78 (2002)). GCC knock out animals have a similar phenotype with hyperplastic crypts and reduced differentiation of secretory lineage cells (Li P, et al., Am J Pathol, 171:1847-58 (2007)).

Compromised luminal integrity is characteristic of intestinal diseases and disorders. Infections, poor blood supply, and autoimmunity have been implicated in inflammatory bowel diseases such as ulcerative colitis. Reactive oxygen species produced by inflammatory leukocytes also contribute to tissue destruction associated with these disorders. Both medications and surgery are used to treat symptoms of ulcerative colitis, however, there is no cure. Since ulcerative colitis cannot be cured by medication, the goals of treatment are typically to induce remission and improve quality of life with a minimum of side effects. Treatment for colitis typically includes anti-inflammatory agents, immunomodulators, or combinations thereof, however not all patients respond to each treatment and some of the treatments are accompanied by serious side effects that impact quality of life for the patient.

High-fat diet or having Crohn's disease, celiac disease, or a history of colonic polyps are associated with an increased incidence of intestinal cancers such as duodenal cancer, ileal cancer, jejunal cancer, small intestine cancer. High-fat diet, a family history of colorectal cancer and polyps, the presence of polyps in the large intestine, and chronic ulcerative colitis are associated with an increased incidence of colon cancer. Reactive oxygen species are also carcinogenic and have been associated with colon cancer. Surgery, radiation, and chemotherapy are the standard of care for treatment of intestinal cancers. Unfortunately, there are no completely effective treatment for many bowel disorders.

Therefore, it is an object of the invention to provide compositions and methods of use thereof for treating inflammatory disorders such as ulcerative colitis with reduced undesirable or harmful side effects.

It is another object of the invention to provide compositions and methods of use for treating cancer, particularly intestinal and colon cancers, with reduced undesirable or harmful side effects.

### SUMMARY OF THE INVENTION

It has been discovered that the cGMP/PKG pathway can be targeted to treat inflammatory bowel diseases, intestinal cancers, and improve the integrity of the luminal surface of the gastrointestinal tract. For example, the pathway can be targeted with compounds that activate PKG, in particular PKG2. Compositions and formulations useful for treating inflammatory bowel diseases, intestinal cancers, and improving the barrier function and increasing the integrity of the luminal surface of the gastrointestinal tract are disclosed. Typically, the composition increases levels of cGMP in the intestinal mucosa or increases the activity of PKGs, preferably PKG2, in the intestinal mucosa. In certain embodiments, the disclosed compositions increase the activity of PKG2 in the intestinal mucosa. Compositions for increasing the level of cGMP in the intestinal mucosa or increasing the activity of PKGs in the intestinal mucosa can include one or more cGMP-specific phosphodiesterase inhibitors or analogs thereof. Preferably, the composition includes a phosphodiesterase type 5 (PDE5) inhibitor. Examples of PDE5 inhibitors include, but are not limited to, avanafil, lodenafil, mirodenafil, sildenafil citrate, tadalafil, vardenafil, udenafil, and icariin. In certain embodiments, the disclosed compositions are targeted to the intestinal mucosa, for example by enteric coating or attachment of an intestinal specific targeting moiety. The biological activity of the disclosed compositions is preferably limited to the intestinal mucosa.

Methods of using the compositions to reduce or alleviate one or more symptoms of an intestinal or bowel disease or disorder, an intestinal cancer, or to improve or increase luminal integrity in the intestine are also disclosed. For example, a method for improving intestinal luminal integrity can include administering to a subject an effective amount of a composition that increases level of cGMP in the intestinal mucosa or increases the activity of a PKG in the intestinal mucosa to increase or improve one or more physiological parameters of intestinal luminal barrier function. Physiological parameters of intestinal luminal barrier function include, but are not limited to, reduced proliferation for example in the intestinal crypts, reduced apoptosis for example at the luminal surface, induction of downstream effectors of PKG mediated signaling, increased DUSP8/10 (MKP5) expression or activation, decreased JNK expression or activation, decreased Sox9 protein levels, inhibition of Sox9 repressive activity, increased activity of an antioxidant, increased expression of one or more genes encoding an antioxidant such as MnSOD, PRDX3, Cat, GSTa3, GSR, GPX1, GPX2, and reduced levels of reduction-oxidation (redox) stress, or combinations thereof.

### BRIEF DESCRIPTION OF THE DRAWINGS

FIG. 1 is a representation of the cGMP/PKG signaling pathway. NO=nitric oxide; GTP=guanosine triphosphate; GMP=guanosine monophosphate; cGMP=cyclical guanosine monophosphate; pGC=“particulate” membrane-bound guanylyl/guanylate cyclase; sGC=soluble cytosolic guanylyl/guanylate cyclase; NOS=nitric oxide synthase; PKG1=Type 1 cGMP-dependent protein kinase; PKG2=Type 2 cGMP-dependent protein kinase; PDE5=phosphodiesterase type 5; ANP=atrial natriuretic peptide; BNP=brain natriuretic peptide.

FIG. 2 is an autoradiograph of an immunoblot showing relative protein levels of PKG1 and PKG2 in the duodenum, jejunum, ilium, proximal colon, and distal colon. Beta-actin is a loading control.

FIGS. 3A, 3B and 3C are bar graphs showing crypt size (×100 mm2) (C); Ki-67/crypt (D); and BrdU/crypt in WT and KO colons. A series of micrographs of tissue sections from an ascending colon harvested from 6-week old WT and KO siblings and stained with H&E, the proliferation marker Ki-67 and DNA incorporation of BrdU were quantified.

FIG. 4A is a bar graph showing the number of TUNEL positive cells/crypt in the proximal colon of WT and KO animals. FIG. 4B is a bar graph showing the number of caspase 3 active cells/crypt in the proximal colon of WT and KO animals. Micrographs of sections of the proximal colon from Prkg2(+/+) (WT, top) and Prkg2(−/−) (KO, bottom) were stained by TUNEL (A) and for active caspase 3 (B) to identify apoptotic cells. Error bars show SEM, *p<0.05, **p<0.001 by two-tailed t-test.

FIG. 5A is a bar graph showing the number of goblet cells/crypt (AB & PAS staining) in the ascending colon of WT and KO animals. FIG. 5B is a bar graph showing the number of Muc2 positive cells/crypt in the ascending colon of WT and KO animals. FIG. 5C is a bar graph showing the number of CgA positive cells/crypt in the ascending colon of WT and KO animals. Micrographs of sections of mucosa of the ascending colon from Prkg2(+/+) (WT, top) and Prkg2(−/−) (KO, bottom) were stained for goblet cell-associated polysaccharides (AB & PAS) (A), Muc2 protein (B), and the enteroendocrine marker chromogranin A (CgA) (C). Error bars show SEM, *p<0.05, **p<0.001 by two-tailed t-test.

FIG. 6A is a panel showing micrographs of an assay for colony formation (Giemsa stained) in LS1747-II-4 and MT29-II-16 PGK2 inducible colon cancer cell lines with (Doxy+cGAMP) and without (Control) doxycycline and 8Br-cGMP induction. FIG. 6B is a bar graph showing colony area (% control) of the assay describe in FIG. 6A for inducible and non-inducible (parental) cells. Control cells are open bars (ctrl), and Doxy+8Br-cGMP cells are filled bars (+). FIG. 6C is a bar graph showing relative growth (MTT/day) for inducible and non-inducible (parental) cells. Control cells are open bars (ctrl), and Doxy+8Br-cGMP cells are filled bars (+). FIG. 6D is a bar graph showing the number of cells (×10⁶) alive (open bars) and dead (filled bars) which are trypan blue positive or trypan blue negative respectively. FIG. 6E is a bar graph showing the % of control of annexin/propidium staining for inducible and non-inducible (parental) cells. Control cells are open bars (ctrl), and Doxy+8Br-cGMP cells are filled bars (+). FIG. 6F is a bar graph showing the % of total cells in Sub-G1, G1, S, and G2 for inducible and non-inducible (parental) cells. Control cells are open bars (ctrl), and Doxy+8Br-cGMP cells are filled bars (+). Error bars show SEM, *p>0.05, two-tailed t-test.

FIG. 7 is a bar graph showing relative luciferase activity in an assay to measure transcription at the CDX2 (open bars) and Muc2 (filled bars) promoters in cells treated with the same levels of 8Br-cGMP and increasing levels of Doxy (ug/ml). Error bars are SEM. *p>0.05, two-tailed t-test.

FIGS. 8A and 8B are bar graphs showing relative Muc2 activity in LS174T-II-4 (A) and HT29 (B) with (cG+Doxy) and without (Control) Doxy+8Br-cGMP induction of PKG2 and with (Sox9) and without (Vector) Sox9 overexpression.

FIG. 9A is a bar graph showing cGMP (pmol/ml) in cell extracts from HCT116 colon cancer cells incubated for 2 hours with cGMP, or PDE5 inhibitors (control, sildenafil, vardenafil, tadalafil as indicated) and analyzed using a competitive enzyme-linked immunosorbent assay (ELISA). Results shown are representative of 3 experiments, and error bars indicate SEM. FIG. 9B is an autoradiograph of an immunoblot showing as a mobility shift of the PKG target vasodilator-stimulated phosphoprotein (VASP) to phosphorylated VASP (P-VASP) of HCT116 colon cancer cells transfected with flag-tagged VASP or VASP and PKG1, and treated for two hours with PDE5 inhibitors (control, sildenafil, vardenafil, tadalafil as indicated).

FIG. 10A is a bar graph showing cGMP (pmol/ml/OD280) in colon mucosa isolated from mouse colon explants incubated for 2 hours PDE5 inhibitors (control, sildenafil, vardenafil, tadalafil as indicated) in vitro and analyzed using a competitive enzyme-linked immunosorbent assay (ELISA). The cGMP levels were standardized for protein content using OD280 of the extracts. FIG. 10B is a bar graph showing cGMP (pmol/ml/OD280) in colon mucosa isolated from control mice, or mice treated with an intraperitoneal injection of vardenafil dissolved in 100 μl of olive oil as the vehicle and analyzed using a competitive enzyme-linked immunosorbent assay (ELISA). Error bars indicate SEM. Asterisks indicate significance relative to control (p<0.05).

FIG. 11A is a bar graph showing goblet cells/crypt in the ileum, ascending colon, and descending colon, of control mice (filled bars) and mice treated with 5 mg/kg vardenafil (IP, b.i.d.) for 7 days. Error bars indicate indicate standard error of the mean (SEM) and *=p<0.05, n=3. FIG. 11B is a line graph showing the goblet cells/crypt in the proximal (prox), middle (mid), and distal (dis) colon of control mice (solid lines) and mice treated with 5 mg/kg vardenafil (IP, b.i.d.) for 7 days. Error bars indicate standard error of the mean (SEM) and *=p<0.05, n=3.

FIG. 12A is a bar graph showing quantitation of cell death in the colon mucosa as the number of TUNEL positive cells/crypt for control mice and mice treated with vardenafil. FIG. 12B is a bar graph showing quantitation of cell proliferation in the colon mucosa as the number of TUNEL positive cells/crypt for control mice and mice treated with vardenafil. Asterisks show statistical significance, p<0.05 of treated relative to control.

FIG. 13 is a line graph plotting the DAI (disease activity index including bleeding and diarrhea) of control mice and mice treated with vardenafil 2 days prior to introducing 5% DSS in a colitis model. Error bars indicate standard deviation and asterisks show statistical difference between vardenafil treated and controls at each day (p<0.05, n=4 for each group).

FIG. 14A is a bar graph showing the number of goblet cells/crypt in the ascending colon of control (open bars, Ctrl) and vardenafil treated (filled bars, yard) wildtype (WT) and Prkg2(−/−) (KO) mice. FIG. 14B is a bar graph showing the number of goblet cells/crypt in the transverse (trans) colon of control (open bars, Ctrl) and vardenafil treated (filled bars, yard) wildtype (WT) and Prkg2(−/−) (KO) mice. FIG. 14C is a bar graph showing the number of TUNEL positive cells/crypt in the ascending colon of control (open bars, Ctrl) and vardenafil treated (filled bars, yard) wildtype (WT) and Prkg2(−/−) (KO) mice.

FIG. 15A is a bar graph showing DCF fluorescence of extracts from colon explants with (cGMP, open bars) or without (control, filled bars) cGMP treatment. FIG. 15B is a bar graph showing DCF fluorescence of extracts from colon explants with (var) or without (control) vardenafil treatment.

FIGS. 16A, 16B, 16C and 16D show that vardenafil increases cGMP levels and regulates cell growth and death in the colon mucosa. FIG. 16A is a bar graph of ileum, proximal and distal tissue versus cGMP (pmol/mg protein). Mice were treated by intraperitoneal injection with PBS in vehicle (ctrl) or Vardenafil in vehicle (Vard.) (equivalence to human dose based on body weight). Colon mucosa was harvested 3 hrs later for analysis of cGMP content with ELISA kit. cGMP level was measured relative to total protein loading. FIG. 16B is a line graph of hours with vardenafil versus cGMP (pmol/mg protein). Proximal colon mucosa was harvested 0, 3, 6 and 8 hours after a single dose of Vardenafil I.P. injection. cGMP level was displayed as time dependent manner. FIG. 16C shows line graphs of proximal, mid, and distal tissue versus Ki-67/crypt in both wild type and knockout mice. Wild type (WT) and Prkg2^{-/-} (KO) mice were continuously treated either without (control, dash lines) or with (Vard. solid lines) Vardenafil for 7 days and the colons were harvested and stained for histological analysis of proliferation (Ki-67). FIG. 16D shows line graphs of proximal, mid and distal tissue versus TUNEL/crypt in both WT and KO mice. Same WT/KO tissues were analyzed for apoptosis (TUNEL). Error bars indicate SEM and asterisks show significance (p<0.05).

FIGS. 17A, 17B, 17C, and 17D are line graphs of ascending, trans, and descending tissue samples versus goblet cells/crypt (17A and B) or CgA/crypt (17C and D). Both Wild type (WT) and Prkg2^{-/-} (KO) mice were either treated without (control, dash lines) or with (Vard. solid lines) Vardenafil in vehicles for 7 days and the colons were harvested for analysis of (A)(B) matured goblet cells and (C)(D) enteroendocrine cells (CgA). Error bars show SEM, N>3, *p<0.05, by two-tailed t-test.

FIGS. 18A and 18B show the regulation of JNK signaling by PKG2 in the colon mucosa. FIG. 18A is a bar graph of goblet cells and CgA versus % of wildtype. FIG. 18B is a bar graph of Ki-67 and TUNEL versus % of wildtype. (A)(B) KO mice were either untreated (white bars), or a JNK inhibitor SP600125 (black bars). After 3 days the colons were harvested for analysis of goblet cells, enteroendocrine cells (CgA), proliferation (Ki-67), and apoptosis (TUNEL). Numbers displayed as percentage of wild type. N=3, *P<0.05

FIGS. 19A, 19B, 19C, and 19D indicate that Vardenafil treatment suppresses DSS-induced colitis in mice. FIG. 19A is a bar graph showing control, cGMP, PKG2, and cG+PKG2 treated samples versus relative Muc2 activity. FIG. 19B is a bar graph showing control and JNK-DN versus relative Muc2 activity. FIG. 19C is a bar graph showing control, vector and PKG2+cG samples versus relative Luc activity. Vector and PKG2+cG samples are from MEK7D. the open bars are c-Jun and the shaded bars are ATF2. FIG. 19D is a bar graph of control, cG+Doxy, and cG+Doxy treated with si-control, si-181, or si-486 samples versus relative Muc2 activity.

FIGS. 20A and 20B indicate that Vardenafil treatment suppresses DSS-induced colitis in mice. FIG. 20A is a line graph of days of treatment versus disease activity index (DAI). Mice were treated without (control, black line) or with Vardenafil (red line) for 2 days prior to adding DSS to the drinking water. The animal weight and stool-composition (diarrhea and bleeding) were recorded daily and are shown as disease activity index. Error bars indicate SD (n=3) and asterisks denote statistical significance (p<0.05, t-test). FIG. 20B is a line graph of proximal, mid, and distal tissue samples versus TUNEL/Crypt. Some mice were sacrificed at Day 5 with DSS for pathological analysis. Descending colons of mice treated with control (Ctrl) and Vardenafil (Vard.) were harvested and stained for apoptosis (TUNEL). Positive cells were subjected to quantification and the quantified cells were graphed. Error bars indicate SEM (n=3) and asterisks denote statistical significance (p<0.05 t-test).

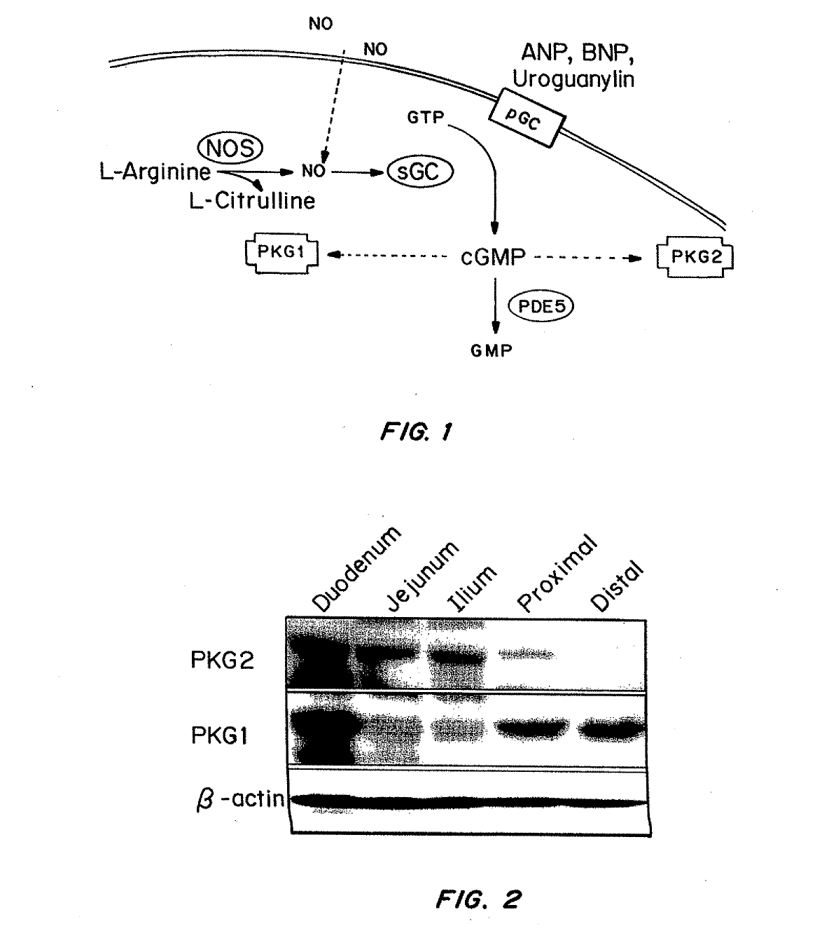

### DETAILED DESCRIPTION OF THE INVENTION

### I. Definitions

The terms “individual,” “subject,” and “patient” are used interchangeably herein, and refer to a mammal, including, but not limited to, rodents, simians, humans, mammalian farm animals, mammalian sport animals, and mammalian pets.

As used herein, the term “treating” includes alleviating, preventing or eliminating one or more symptoms associated with inflammatory bowel disorders, cancer, diarrhea, or other indications disclosed herein.

The term “reduce”, “inhibit” or “decrease” are used relative to a control. One of skill in the art would readily identify the appropriate control to use for each experiment. For example a decreased response in a subject or cell treated with a compound is compared to a response in subject or cell that is not treated with the compound.

As used herein, the phrase “increased activity” of a protein means increased biological activity of the protein, for example enzymatic activity. Protein activity can include signal transduction involving the protein. Increased activity may or may not include an increased amount of the protein, or increased gene expression. For example, increased activity of a kinase may include an increase in phosphorylation of targets of the kinase. If a protein is activated by phosphorylation, increased activity may include an increase in the amount of phosphorylated protein or a decrease in the amount on non-phosphorylated protein.

As used herein, the phrase “decreased activity” of a protein means decreased biological activity of the protein or decreased signal transduction involving the protein. Decreased activity may or may not include a decreased amount of the protein, or decreased gene expression. For example, decreased activity of a kinase may include a decrease in phosphorylation of targets of the kinase. If a protein is activated by phosphorylation, decreased activity may include a decrease in the amount of phosphorylated protein or an increase in the amount of non-phosphorylated protein.

As used herein the terms “gastrointestinal tract” and “gut” refer to the digestive tract from the stomach to the anus.

As used herein the terms “bowel,” “intestine,” “intestines,” and “intestinal” can refer to the small intestine, the large intestine, or combinations thereof. The small intestine includes the duodenum, jejunum, and the ileum. The large intestine includes the cecum, the vermiform appendix, the colon, and the rectum. The colon includes the ascending colon, the traverse colon, the descending colon, and the sigmoid fixture.

As used herein the term “intestinal mucosa” means glandular epithelium and muscularis mucosa.

As used herein, the term “phosphodiesterase inhibitor” refers to a compound that inhibits one or more phosphodiesterases (PDE) “Inhibiting” a phosphodiesterase means to partially or completely reduce, or inhibit the PDE from degrading intracellular second messengers cyclic adenosine monophosphate (cAMP), cyclic guanosine monophosphate (cGMP) or combinations thereof.

As used herein, the term “phosphodiesterase 5 inhibitor”, or “PDE5 inhibitor” means a compound that blocks the action of phosphodiesterase type 5, a cGMP specific phosphodiesterase.

“Pharmaceutically acceptable”, as used herein, refers to compounds, materials, compositions, and dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), more preferably 20 or fewer carbon atoms, more preferably 12 or fewer carbon atoms, and most preferably 8 or fewer carbon atoms. Likewise, preferred cycloalkyls have from 3-10 carbon atoms in their ring structure, and more preferably have 5, 6 or 7 carbons in the ring structure. The ranges provided above are inclusive of all values between the minimum value and the maximum value.

The alkyl groups may also be substituted with one or more groups including, but not limited to, halogen, hydroxy, amino, thio, ether, ester, carboxy, oxo, and aldehyde groups. The alkyl groups may also contain one or more heteroatoms within the carbon backbone. Preferably the heteroatoms incorporated into the carbon backbone are oxygen, nitrogen, sulfur, and combinations thereof. In certain embodiments, the alkyl group contains between one and four heteroatoms.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

“Prodrug”, as used herein, refers to a pharmacological substance (drug) which is administered in an inactive (or significantly less active) form. Once administered, the prodrug is metabolized in the body (in vivo) into the active compound.

The terms “Analog” and “Derivative” are used herein interchangeably, and refer to a compound having a structure similar to that a parent compound, but varying from the parent compound by a difference in one or more certain components. The analog or derivative can differ from the parent compound in one or more atoms, functional groups, or substructures, which are replaced with other atoms, groups, or substructures. An analog or derivative can be imagined to be formed, at least theoretically, from the parent compound via some chemical or physical process.

### II. Compositions

The intestinal tract is a diverse microenvironment which is home to more 500 species of bacteria. A single layer of luminal epithelium is the only barrier separating both symbiotic bacteria, pathogens, and other luminal antigens from the underlying immune cells. Therefore, a healthy and intact epithelial barrier is important in the prevention infection and inflammation.

As shown in FIG. 1, the canonical activation route for PKG is by elevations in cytosolic cGMP (Browning, et al., Future Med. Chem., 2(1):65-80 (2010)). cGMP levels are regulated by activation of soluble guanylyl cyclase (sGC) and membrane bound “particulate” guanylyl cyclase receptors (pGC) with intracellular guanylyl cyclase activity. Dysregulated cGMP signaling can contribute to cancer and inflammatory diseases.

It has been discovered that the cGMP/PKG pathway can be targeted to treat inflammatory bowel diseases, intestinal cancers, and improve the integrity of the luminal surface of the gastrointestinal tract. Compositions useful in the methods described in detail below typically increase the level of cGMP in the intestinal mucosa or increase the activity of a PKG, preferably PKG2, in the intestinal mucosa.

A. Active Agents

The disclosed compositions can include small molecules, therapeutic polypeptides, antibodies, inhibitory nucleic acids, or combinations thereof. In some embodiments, the therapeutic polypeptide or inhibitory nucleic acid is expressed from an expression vector including a nucleic acid sequence that encodes the therapeutic polypeptide or inhibitory nucleic acid.

In some embodiments, the composition increases the activity of PKG1. PKG1 activates FoxO signaling in the colon mucosa leading to inhibition of beta-catenin/TCF signaling. PKG1 also activates antioxidant gene expression in the colon mucosa and is believed to create a barrier to redox stress. PKG1 also blocks colon cancer cell viability in hypoxic environments.

In some embodiments, the composition increases the activity of PKG2. PKG2 promotes differentiation and reduces proliferation in the colon mucosa. It is believed that PKG2 may promote differentiation through inhibition of Sox9 signaling. Therefore, in some embodiments, the composition inhibits Sox9 signaling. In some embodiments the composition increases the activity of PKG2 without increasing the activity of PKG1.

In some embodiments the composition increases the activity of a soluble or particulate guanylyl cyclase.

Typically, PKG is activated by elevations in cytosolic cGMP. Therefore, in some embodiments, the composition increases the bioactive pool of cGMP. In some embodiments the level of bioactive cGMP is increased by adding additional cGMP. In some embodiments the level of bioactive cGMP is increased by reducing breakdown, reduction, degradation, or turnover of cGMP. For example, suitable compositions for use with the methods disclosed herein include agents that block breakdown or reduction in cytosolic levels of cGMP.

Phosphodiesterases such as the cGMP-specific phosphodiesterase type 5 (PDE5) inhibitor, limit signaling through cGMP pathway by hydrolyzing cGMP to GMP. Therefore, compositions that activate or increase PKG activity include phosphodiesterase inhibitors. In one embodiment, the composition contains a cGMP-specific phosphodiesterase inhibitor, or pharmaceutically acceptable analog, prodrug, salt, solvate, or clathrate thereof.

The composition can contain one or more cGMP-specific phosphodiesterase inhibitors. In preferred embodiments, the composition contains one or more phosphodiesterase 5 (PDE5) inhibitors. Suitable PDE5 inhibitors are known in the art, and include avanafil, lodenafil, mirodenafil, sildenafil, tadalafil, vardenafil, udenafil, zaprinast, and icariin.

In some embodiments, the PDE5 inhibitor is a 2-phenyl-substituted imidazotriazinone defined by Formula I

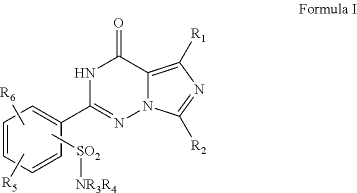

wherein

R₁ is hydrogen or a straight-chain or branched alkyl group having between 1 and 6 carbon atoms,

R₂ is a straight-chain or branched alkyl having between 1 and 6 carbon atoms,

R₃ and R₄ are, independently, hydrogen, a straight-chain or branched alkenyl or alkoxy group having up to 8 carbon atoms, or a straight-chain or branched alkyl chain having up to 10 carbon atoms which is optionally interrupted by an oxygen atom and which optionally contains one or more substituents selected from trifluoromethyl, trifluoromethoxy, hydroxyl, halogen, carboxyl, benzyloxycarbonyl, straight-chain or branched alkoxycarbonyl having up to 6 carbon atoms, and/or by radicals of the formulae —SO₃H, -(A)₃, —NR₇R₈, —P(O)(OR₁₀)(OR₁₁),

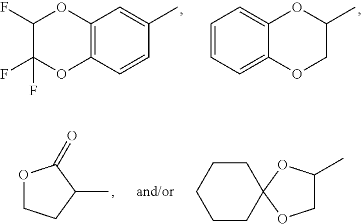

wherein

a and b are, independently, either 0 or 1,

A represents a radical CO or SO₂,

R₇, R₇, R₈, and R_{8′} are independently hydrogen, a cycloalkyl group having between 3 and 8 carbon atoms, an aryl group having between 6 and 10 carbon atoms, a 5- or 6-membered unsaturated, partially unsaturated or saturated, optionally benzo-fused heterocycle having up to 3 heteroatoms selected from S, N and O, where the abovementioned ring systems can optionally contain one or more substituents selected from hydroxyl, nitro, trifluoromethyl, trifluoromethoxy, carboxyl, halogen, a straight-chain or branched alkoxy or alkoxycarbonyl group having between 1 and 6 carbon atoms, and/or by a group represented by formula —(SO₂)_{c}—NR₁₂R₁₃, in which c is either 0 or 1, and R₁₂ and R₁₃ are independently hydrogen or straight-chain or branched alkyl group having between 1 and 6 carbon atoms, or

R₇, R₇, R₈, and R_{8′} each represent straight-chain or branched alkoxy group having between 1 and 6 carbon atoms, or a straight-chain or branched alkyl group having between 1 and 8 carbon atoms which optionally contains one or more substituents selected from hydroxyl, halogen, and aryl group having between 6 and 10 carbon atoms, a straight-chain or branched alkoxy or alkoxycarbonyl group having between 1 and 6 carbon atoms, and/or a group represented by formula —(CO)_{d}—NR₁₄R₁₅, in which R₁₄ and R₁₅ are identical or different and each represents hydrogen or straight-chain or branched alkyl having up to 4 carbon atoms, and d represents a number 0 or 1, or

R₇ and R₈ and/or R₇ and R_{8′}, together with the nitrogen atom to which they are attached, form a 5- to 7-membered saturated heterocycle which may optionally contain one or more additional heteroatoms selected from S and O, or a radical of the formula —NR₁₆, in which R₁₆ represents hydrogen, an aryl group having between 6 and 10 carbon atoms, benzyl, a 5- to 7-membered aromatic or saturated heterocycle having up to 3 heteroatoms selected from S, N and O optionally substituted by a methyl group, or a straight-chain or branched alkyl group having between 1 and 8 carbon atoms optionally substituted by hydroxyl,

R₉ represents an aryl group having between 6 and 10 carbon atoms, or a straight-chain or branched alkyl group having between 1 and 6 carbon atoms,

R₁₀ and R₁₁ are identical or different and each represents hydrogen or straight-chain or branched alkyl group having between 1 and 6 carbon atoms, and/or the alkyl chain listed above under R₃/R₄ is optionally substituted by cycloalkyl group having 3 to 8 carbon atoms, an aryl group having between 6 and 10 carbon atoms, or by a 5- to 7-membered partially unsaturated, saturated, or unsaturated, optionally benzo-fused heterocycle which may contain up to 4 heteroatoms selected from S, N and O, or a radical of the formula —NR₁₇, in which R₁₇ represents hydrogen, hydroxyl, formyl, trifluoromethyl, a straight-chain or branched acyl or alkoxy group having between 1 and 6 carbon atoms, or a straight-chain or branched alkyl group having between 1 and 6 carbon atoms which is optionally substituted by one or more substituents selected from hydroxyl and straight-chain or branched alkoxy group having between 1 and 6 carbon atoms, and where aryl and the heterocycle are optionally substituted with one or more substituents selected from nitro, halogen, —SO₃H, a straight-chain or branched alkyl or alkoxy group having between 1 and 6 carbon atoms, hydroxyl, trifluoromethyl, trifluoromethoxy, and/or by a radical of the formula —SO₂—NR₁₈R₁₉, in which R₁₈ and R₁₉ are individually hydrogen or a straight-chain or branched alkyl group having between 1 and 6 carbon atoms, and/or

R₃ or R₄ represents a group of the formula —NR₂OR₂₁, in which R₂₀ and R₂₁ are individually hydrogen or a straight-chain or branched alkyl group having between 1 and 6 carbon atoms, and/or

R₃ or R₄ represents adamantyl, or represents radicals of the formulae

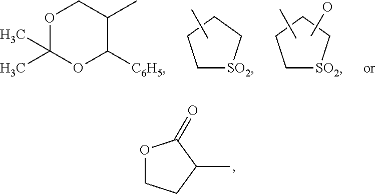

or represents cycloalkyl group having between 3 and 8 carbon atoms, an aryl group having between 6 and 10 carbon atoms, or represents a 5- to 7-membered partially unsaturated, saturated or unsaturated, optionally benzo-fused heterocycle which may contain up to 4 heteroatoms selected from S, N and O, or a radical of the formula —NR₂₂, in which R₂₂ represents hydrogen, an aryl group having between 6 and 10 carbon atoms, benzyl, a 5- to 7-membered aromatic or saturated heterocycle having up to 3 heteroatoms selected from S, N and O optionally substituted by a methyl group, a straight-chain or branched alkyl group having between 1 and 8 carbon atoms optionally substituted by hydroxyl, or represents carboxyl, formyl or straight-chain or branched acyl group having between 1 and 6 carbon atoms, and where the cycloalkyl, aryl and/or the heterocycle are optionally substituted with one or more substituents selected from halogen, triazolyl, trifluoromethyl, trifluoromethoxy, carboxyl, straight-chain or branched acyl or alkoxycarbonyl having in each case between 1 and 6 carbon atoms, nitro, and/or by groups of the formulae —SO₃H, —OR₂₃, (SO₂)ₑNR₂₄R₂₅, —P(O)(OR₂₆)(OR₂₇), in which e represents a number 0 or 1, R₂₃ represents a radical of the formula

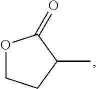

o represents a cycloalkyl group between 3 and 7 carbon atoms, or represents hydrogen or straight-chain or branched alkyl group having between 1 and 6 carbon atoms, optionally substituted by cycloalkyl group having between 3 and 7 carbon atoms, benzyloxy, tetrahydropyranyl, tetrahydrofuranyl, a straight-chain or branched alkoxy or alkoxycarbonyl group having between 1 and 6 carbon atoms, carboxyl, benzyloxycarbonyl or phenyl, which may be optionally substituted by one or more substituents selected from a straight-chain or branched alkoxy group having between 1 and 6 carbon atoms, hydroxyl, halogen, and/or an alkyl group which is optionally substituted by radicals of the formulae —CO—NR₂₈R₂₉ or —CO—R₃₀, in which R₂₈ and R₂₉ are independently hydrogen or a straight-chain or branched alkyl group having between 1 and 8 carbon atoms, or R₂₈ and R₂₉, together with the nitrogen atom to which they are attached, form a 5- to 7-membered heterocycle which may optionally contain one or more additional heteroatoms selected from S, N, and O, and R₃₀ represents phenyl or adamantyl, R₂₄ and R₂₅ have the meanings of R₁₈ and R₁₉ given above, R₂₆ and R₂₇ have the meanings of R₁₀ and R₁₁ given above, and/or the cycloalkyl, aryl and/or the heterocycle are optionally substituted by straight-chain or branched alkyl group having between 1 and 8 carbon atoms which is optionally substituted by one or more substituents selected from hydroxyl, carboxyl, a 5- to 7-membered heterocycle having up to 3 heteroatoms selected from S, N and O, or by groups of the formula —SO₂—R₃₁, P(O)(OR₃₂)(OR₃₃), or —NR₃₄R₃₅, in which R₃₁ is hydrogen or has the meaning of R₉ given above, R₃₂ and R₃₃ have the meanings of R₁₀ and R₁₁ given above, R₃₄ and R₃₅ are independently hydrogen, or a straight-chain or branched alkyl group between 1 and 8 carbon atoms and optionally substituted one or more substituents selected from hydroxyl or a straight-chain or branched alkoxy group having between 1 and 6 carbon atoms, or R₃₄ and R₃₅, together with the nitrogen atom to which they are attached, form a 5- to 7-membered saturated heterocycle which may contain one or more additional heteroatoms selected from S and O, or a radical of the formula —NR₃₆, in which R₃₆ represents hydrogen, hydroxyl, a straight-chain or branched alkoxycarbonyl group having between 1 and 8 carbon atoms, or a straight-chain or branched alkyl group having between 1 and 6 carbon atoms, optionally substituted by one or more substituents selected from hydroxyl, or

R₃ and R₄, together with the nitrogen atom to which they are attached, form a 5- to 7-membered unsaturated or saturated or partially unsaturated, optionally benzo-fused heterocycle, which may optionally contain up to 3 heteroatoms selected from S, N and O, or a radical of the formula —NR₃₇, in which R₃₇ represents hydrogen, hydroxyl, formyl, trifluoromethyl, straight-chain or branched acyl group, alkoxy group, or alkoxycarbonyl group having in each case between 1 and 6 carbon atoms, or represents straight-chain or branched alkyl group having between 1 and 6 carbon atoms, optionally substituted with one or more substituents selected from hydroxyl, trifluoromethyl, carboxyl, a straight-chain or branched alkoxy or alkoxycarbonyl group having between 1 and 6 carbon atoms, or by groups of the formula -(D)_{f}-NR₃₈R₃₉, —CO—(CH₂)_{g}—O—CO—R₄₀, —CO—(CH₂)ₕ—OR₄₁, or —P(O)(OR₄₂)(OR₄₃), in which g and h are independently a number between 1 and 4, f represents a number 0 or 1, D represents a group of the formula —CO or —SO₂, R₃₈ and R₃₉ independently have the meanings of R₇ and R₈ given above, R₄₀ represents a straight-chain or branched alkyl group having between 1 and 8 carbon atoms, R₄₁ represents a straight-chain or branched alkyl group having between 1 and 8 carbon atoms, R₄₂ and R₄₃ are independently hydrogen, or a straight-chain or branched alkyl group having between 1 and 6 carbon atoms, or R₃₇ represents a radical of the formula —(CO)ᵢ-E, in which i represents a number 0 or 1, E represents a cycloalkyl group between 3 and 7 carbon atoms or benzyl, an aryl group having between 6 and 10 carbon atoms, or a 5- to 7-membered aromatic heterocycle having between 1 and 4 heteroatoms selected from S, N and O, where the above-mentioned ring systems are optionally substituted by one or more substituents selected from nitro, halogen, —SO₃H, a straight-chain or branched alkoxy group having between 1 and 6 carbon atoms, hydroxyl, trifluoromethyl, trifluoromethoxy, or by a radical of the formula —SO₂—NR₄₄R₄₅, in which R₄₄ and R₄₅ have the meanings of R₁₈ and R₁₉ given above, or E represents radicals of the formulae

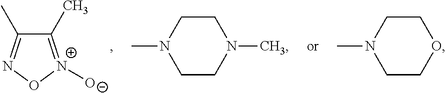

and the heterocycles listed under R₃ and R₄, formed together with the nitrogen atom, and optionally substituted by one or more substituents, if appropriate also geminally, selected from the group consisting of hydroxyl, formyl, carboxyl, a straight-chain or branched acyl or alkoxycarbonyl group having between 1 and 6 carbon atoms, nitro, and groups of the formulae —P(O)(OR₄₆)(OR₄₇),

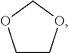

═NR₄₈, or —(CO)ⱼNR₄₉R₅₀, in which R₄₆ and R₄₇ independently have the meanings of R₁₀ and R₁₁, R₄₈ represents hydroxyl or straight-chain or branched alkoxy group having between 1 and 6 carbon atoms, j represents a number 0 or 1, and R₄₉ and R₅₀ independently have the meanings of R₁₄ and R₁₅, and/or the heterocycle listed under R₃ and R₄, which is formed together with the nitrogen atom, is optionally substituted by a straight-chain or branched alkyl group having between 1 and 8 carbon atoms which is optionally by one or more substituents selected from hydroxyl, halogen, carboxyl, a cycloalkyl or cycloalkyloxy group having in each case between 3 to 8 carbon atoms, a straight-chain or branched alkoxy or alkoxycarbonyl group having in each case between 1 and 6 carbon atoms, or by a radical of the formula —SO₃H, —NR₅₁R₅₂ or P(O)OR₅₃OR₅₄, in which R₅₁ and R₅₂ are independently hydrogen, phenyl, carboxyl, benzyl, or a straight-chain or branched alkyl or alkoxy group having between 1 and 8 carbon atoms, R₅₃ and R₅₄ independently have the meanings of R₁₀ and R₁₁, and/or the alkyl group is optionally substituted by aryl having between 6 and 10 carbon atoms which may be optionally substituted with one or more substituents selected from halogen, hydroxyl, a straight-chain or branched alkoxy group having between 1 and 6 carbon atoms, or by a group of the formula —NR_{51′}R_{52′} in which R_{51′} and R_{52′} have the meanings of R₅₁ and R₅₂, and/or the heterocycle listed under R₃ and R₄, which is formed together with the nitrogen atom, is optionally substituted by an aryl group having between 6 and 10 carbon atoms or by a 5- to 7-membered saturated, partially unsaturated or unsaturated heterocycle containing one or more heteroatoms selected from S, N and O, optionally attached via a nitrogen function, where the ring systems may be substituted by hydroxyl or by a straight-chain or branched alkyl or alkoxy group between 1 and 8 carbon atoms, or R₃ and R₄, together with the nitrogen atom to which they are attached, form radicals of the formulae

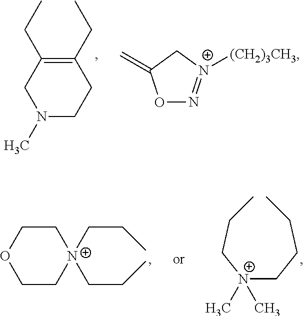

and

R₅ and R₆ are, independently, hydrogen, hydroxyl, a straight-chain or branched alkyl group having between 1 and 8 carbon atoms, or a straight-chain or branched alkoxy group having between 1 and 8 carbon atoms,

or a pharmaceutically acceptable analog, salt, solvate, N-oxide, or isomeric form thereof.

2-phenyl-substituted imidazotriazinone PDE5 inhibitors, including those defined by Formula I, are known in the art. See, for example, U.S. Pat. No. 6,362,178. In a preferred embodiment, the PDE5 inhibitor is vardenafil (more preferably vardenafil hydrochloride), the structure of which is shown below.

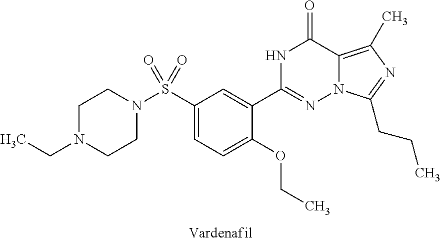 [SMILES: CCCc1nc(C)c2c(=O)[nH]c(-c3cc(S(=O)(=O)N4CCN(CC)CC4)ccc3OCC)nn12]

In some embodiments, the PDE5 inhibitor is a pyrazolo[4,3-d]pyrimidin-7-one defined by Formula IIa

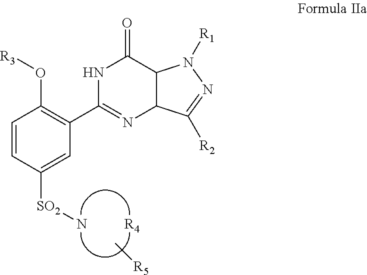

wherein,

R₁ is hydrogen, a C₁-C₈ alkyl group or a C₃-C₈ alkyl group optionally substituted with one or more substituents selected from halogen, hydroxyl, CF₃, nitrile, alkyl, cycloalkyl, aryl, heteroaryl, ester, ether, amine, amide, urea, carbamate, and thioether, or a C₁-C₈ perfluoroalkyl group,

R₂ is hydrogen, a C₁-C₈ alkyl or alkoxy group or a C₃-C₈ cycloalkyl group optionally substituted with one or more substituents selected from halogen, hydroxyl, CF₃, nitrile, alkyl, cycloalkyl, aryl, heteroaryl, ester, ether, amine, amide, urea, carbamate, and thioether, or a C₁-C₈ perfluoroalkyl group,

R₃ is a C₁-C₈ alkyl group or a C₃-C₈ alkenyl or alkynyl group optionally substituted with one or more substituents selected from halogen, hydroxyl, CF₃, nitrile, alkyl, cycloalkyl, aryl, heteroaryl, ester, ether, amine, amide, urea, carbamate, and thioether, or a C₁-C₈ perfluoroalkyl group,

R₄, taken together with the nitrogen atom to which it is attached, forms a pyrrolidinyl, piperidino, morpholino, or 4-N—(R₆)-piperazinyl group;

R₅ is hydrogen, a C₁-C₈ alkyl or C₁-C₈ alkoxy group optionally substituted with one or more substituents selected from halogen, hydroxyl, CF₃, nitrile, alkyl, cycloalkyl, aryl, heteroaryl, ester, ether, amine, amide, urea, carbamate, and thioether, —NR₇R₈, or —CONR₇R₈,

R₆ is hydrogen, a C₁-C₈ alkyl group optionally substituted with one or more substituents selected from halogen, hydroxyl, CF₃, nitrile, alkyl, cycloalkyl, aryl, heteroaryl, ester, ether, amine, amide, urea, carbamate, and thioether, —CONR₇R₈, —CSNR₇R₈, or —C(NH)NR₇R₈, and

R₇ and R₈ are each independently hydrogen, or a C₁-C₈ alkyl group optionally substituted with one or more substituents selected from halogen, hydroxyl, CF₃, nitrile, alkyl, cycloalkyl, aryl, heteroaryl, ester, ether, amine, amide, urea, carbamate, and thioether,

or a pharmaceutically acceptable analog, salt, solvate, N-oxide, or isomeric form thereof.

In some embodiments, the PDE5 inhibitor is a pyrazolo[4,3-d]pyrimidin-7-one defined by Formula IIb

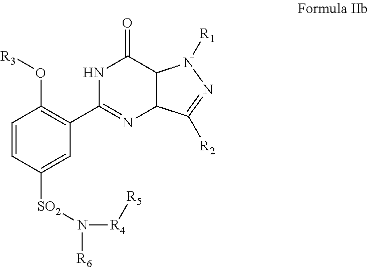

wherein,

R₁ is hydrogen, a C₁-C₈ alkyl group or a C₃-C₈ alkyl group optionally substituted with one or more substituents selected from halogen, hydroxyl, CF₃, nitrile, alkyl, cycloalkyl, aryl, heteroaryl, ester, ether, amine, amide, urea, carbamate, and thioether, or a C₁-C₈ perfluoroalkyl group,

R₂ is hydrogen, a C₁-C₈ alkyl or alkoxy group or a C₃-C₈ cycloalkyl group optionally substituted with one or more substituents selected from halogen, hydroxyl, CF₃, nitrile, alkyl, cycloalkyl, aryl, heteroaryl, ester, ether, amine, amide, urea, carbamate, and thioether, or a C₁-C₈ perfluoroalkyl group,

R₃ is a C₁-C₈ alkyl group or a C₃-C₈ alkenyl or alkynyl group optionally substituted with one or more substituents selected from halogen, hydroxyl, CF₃, nitrile, alkyl, cycloalkyl, aryl, heteroaryl, ester, ether, amine, amide, urea, carbamate, and thioether, or a C₁-C₈ perfluoroalkyl group,

R₄ is a C₁-C₈ alkyl or C₁-C₈ cycloalkyl group optionally substituted with one or more substituents selected from halogen, hydroxyl, CF₃, nitrile, alkyl, cycloalkyl, aryl, heteroaryl, ester, ether, amine, amide, urea, carbamate, and thioether, or —CONR₇—,

R₅ is a C₃-C₈ cycloalkyl group optionally substituted with one or more substituents selected from halogen, hydroxyl, CF₃, nitrile, alkyl, cycloalkyl, aryl, heteroaryl, ester, ether, amine, amide, urea, carbamate, and thioether, a 5- to 7-membered heterocyclyl group containing between one and three heteroatoms selected from O, N, and S and optionally substituted with one or more substituents selected from halogen, hydroxyl, CF₃, nitrile, alkyl, cycloalkyl, aryl, heteroaryl, ester, ether, amine, amide, urea, carbamate, and thioether, or a C₅-C₁₀ aryl or heteroaryl group, the heteroaryl group containing between one and three heteroatoms selected from O, N, and S, both optionally substituted with one or more substituents selected from halogen, hydroxyl, CF₃, nitrile, alkyl, cycloalkyl, aryl, heteroaryl, ester, ether, amine, amide, urea, carbamate, and thioether, and

R₆ and R₇ are each independently hydrogen, or a C₁-C₈ alkyl group optionally substituted with one or more substituents selected from halogen, hydroxyl, CF₃, nitrile, alkyl, cycloalkyl, aryl, heteroaryl, ester, ether, amine, amide, urea, carbamate, and thioether,

or a pharmaceutically acceptable analog, salt, solvate, N-oxide, or isomeric form thereof.

Pyrazolo[4,3-d]pyrimidin-7-one PDE5 inhibitors, including those defined by Formulae IIa and IIb, are known in the art. See, for example, U.S. Pat. No. 5,250,534. In certain embodiments, the PDE5 inhibitor is sildenafil (more preferably sildenafil citrate), udenafil, or mirodenafil, the structures of which are shown below.

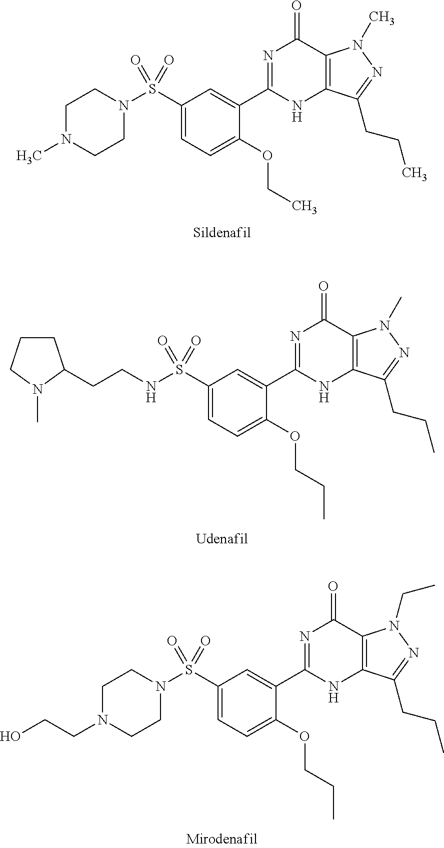 [SMILES: CCCc1nn(C)c2c(=O)nc(-c3cc(S(=O)(=O)N4CCN(C)CC4)ccc3OCC)[nH]c12 ; CCCOc1ccc(S(=O)(=O)NCCC2CCCN2C)cc1-c1nc(=O)c2c([nH]1)c(CCC)nn2C ; CCCOc1ccc(S(=O)(=O)N2CCN(CCO)CC2)cc1-c1nc(=O)c2c([nH]1)c(CCC)nn2CC]

Other suitable PDE-5 inhibitors are also known in the art. See, for example, U.S. Pat. Nos. 5,859,006, 6,140,329, 6,548,490, and 6,821,978, U.S. Published Patent Application Nos. US 2003/0139384 and US 2007/0122355, and PCT Publication Nos. WO 94/28902 and WO 96/16644. Examples of other suitable PDE5 inhibitors include tadalafil, avanafil, lodenafil, icariin, and zaprinast, the structures of which are shown below.

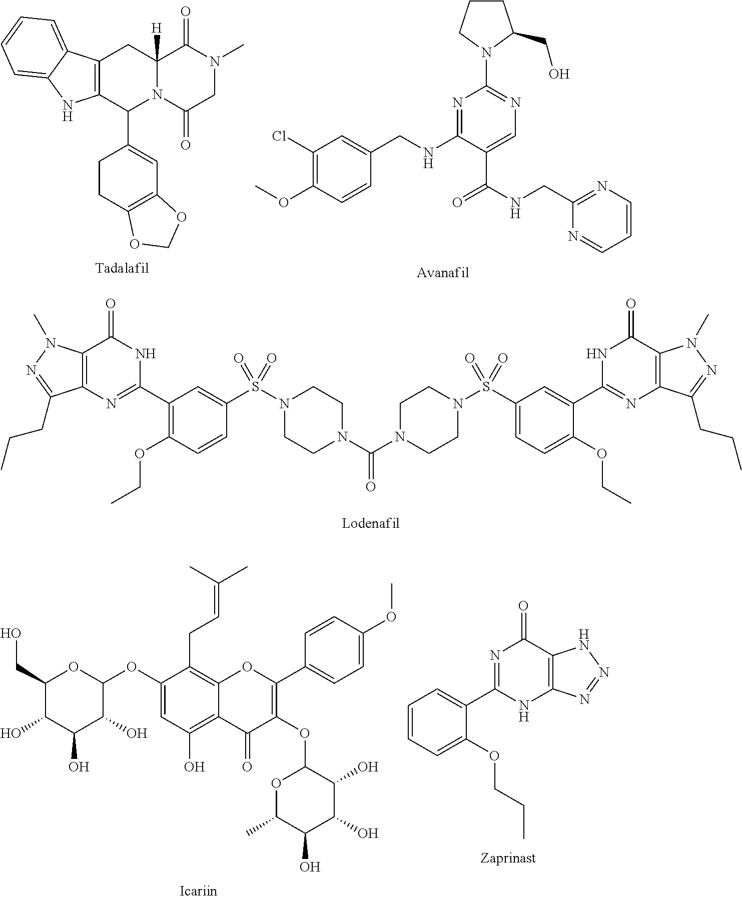 [SMILES: CN1CC(=O)N2C(C3=CC4=C(CC3)OCO4)c3[nH]c4ccccc4c3C[C@@H]2C1=O ; COc1ccc(CNc2nc(N3CCC[C@H]3CO)ncc2C(=O)NCc2ncccn2)cc1Cl ; CCCc1nn(C)c2c(=O)[nH]c(-c3cc(S(=O)(=O)N4CCN(C(=O)N5CCN(S(=O)(=O)c6ccc(OCC)c(-c7nc8c(CCC)nn(C)c8c(=O)[nH]7)c6)CC5)CC4)ccc3OCC)nc12 ; COc1ccc(-c2oc3c(CC=C(C)C)c(OC4O[C@H](CO)[C@@H](O)[C@H](O)[C@H]4O)cc(O)c3c(=O)c2OC2O[C@@H](C)[C@H](O)[C@@H](O)[C@H]2O)cc1 ; CCCOc1ccccc1-c1nc(=O)c2[nH]nnc2[nH]1]

Compositions can also contain an analog of a known PDE5 inhibitor, such as an analog of avanafil, lodenafil, mirodenafil, sildenafil, tadalafil, vardenafil, udenafil, zaprinast, or icariin. Analogs of PDE5 inhibitors include structurally related compounds which display similar pharmacological activity when administered to a patient in need thereof.

Compositions can also contain a pharmaceutically acceptable prodrug of a PDE5 inhibitor or a PDE5 inhibitor analog. Prodrugs are compounds that, when metabolized in vivo, undergo conversion to compounds having the desired pharmacological activity. Prodrugs can be prepared by replacing appropriate functionalities present in a PDE5 inhibitor or PDE5 inhibitor analog with “pro-moieties” as described, for example, in H. Bundgaar, Design of Prodrugs (1985). Examples of preferred prodrugs include ester derivatives, ether derivatives, amide derivatives, pegylated derivatives, phosphate derivatives, and sulfate derivatives of the PDE5 inhibitor and inhibitor analogs described herein, as well as their pharmaceutically acceptable salts.

Prodrugs of PDE5 inhibitors and PDE5 inhibitor analogs may be prepared using standard procedures known in the art. Suitable synthetic methods for the preparation of prodrugs including ester derivatives, ether derivatives, amide derivatives, pegylated derivatives, phosphate derivatives, and sulfate derivatives, are described, for example, in M. Smith and J. March, Advanced Organic Chemistry; Reactions, Mechanisms and Structure, 5th Ed. (New York: Wiley-Interscience, 2001). The preparation of esters derivatives typically involves the functionalization of hydroxyl and/or carboxyl groups which may be present within the molecular structure of the drug. The esters are typically acyl-substituted derivatives of free alcohol groups (i.e., moieties which are derived from carboxylic acids of the formula RCOOH where R is alkyl, preferably a C₁-C₁₈ alkyl group, optionally containing one or more heteroatoms selected from O, S, and N, and optionally substituted by one or more substituents selected from halogen, hydroxyl, CF₃, nitrile, alkyl, cycloalkyl, aryl, heteroaryl, ester, ether, amine, amide, urea, carbamate, sulfate, phosphate, and thioether). Esters can be reconverted to the free acids, if desired, by using conventional hydrogenolysis or hydrolysis procedures. Amides may be prepared from esters, using suitable amine reactants. Amides may also be prepared from an anhydride or an acid chloride by reaction with ammonia or a lower alkyl amine. For further discussions of prodrugs, see, for example, T. Higuchi and V. Stella “Pro-drugs as Novel Delivery Systems,” ACS Symposium Series 14 (1975) and E. B. Roche ed., Bioreversible Carriers in Drug Design (1987).

Compositions can also contain a pharmaceutically acceptable salt of a PDE5 inhibitor or a PDE5 inhibitor analog. Generally, pharmaceutically acceptable salts can be prepared by reaction of the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found, for example, in Remington's Pharmaceutical Sciences, 20th ed., Lippincott Williams & Wilkins, Baltimore, Md., 2000, p. 704.

Suitable acids for preparing acid addition salts include organic acids and inorganic acids. Organic acids include, but are not limited to, acetic acid, propionic acid, glycolic acid, pyruvic acid, oxalic acid, malic acid, malonic acid, succinic acid, maleic acid, fumaric acid tartaric acid, citric acid, benzoic acid, cinnamic acid, mandelic acid, methanesulfonic acid, ethanesulfonic acid, p-toluenesulfonic acid, or salicylic acid. Inorganic acids include, but are not limited to, hydrochloric acid, hydrobromic acid, sulfuric acid, nitric acid, or phosphoric acid. Conversely, basic salts of acid moieties which may be present on a phosphodiesterase inhibitor molecule are prepared in a similar manner using a pharmaceutically acceptable base. Suitable bases include, but are not limited to, sodium hydroxide, potassium hydroxide, ammonium hydroxide, calcium hydroxide, or trimethylamine.

Compositions can also contain a pharmaceutically acceptable solvate of a PDE5 inhibitor or a PDE5 inhibitor analog. Solvates of PDE5 inhibitors or PDE5 inhibitor analogs include molecular complexes formed between a PDE5 inhibitor or a PDE5 inhibitor analog and one or more pharmaceutically acceptable solvent molecules (e.g., ethanol). The term “hydrate” refers to solvates in which the pharmaceutically acceptable solvent is water. A currently accepted classification system for solvates of organic compounds is one that distinguishes between isolated site, channel, and metal-ion coordinated solvates and hydrates. See, for example, K. R. Morris (H. G. Brittain ed.) Polymorphism in Pharmaceutical Solids, Marcel Dekker Inc., New York, N.Y. (1995). Isolated site solvates are solvates in which the solvent molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent is tightly bound, the solvate will have a well-defined stoichiometry independent of ambient humidity. However, when the solvent is weakly bound, as in channel solvates and in hygroscopic compounds, the solvent content will depend on humidity and drying conditions. In such cases, the solvate will generally incorporate solvent molecules in a non-stoichiometric ratio.

Compositions can also contain a pharmaceutically acceptable clathrate of a PDE5 inhibitor or a PDE5 inhibitor analog. Clathrates are drug-host inclusion complexes formed when a drug is associated with or in a host molecule or molecules in stoichiometric ratio. For example, a PDE5 inhibitor or a PDE5 inhibitor analog can form an inclusion complex with a cyclodextrin or other host molecule.

Formulations can also contain pharmaceutically acceptable co-crystals of a PDE5 inhibitor or a PDE5 inhibitor analog. Co-crystals are crystalline complexes of two or more molecular constituents, one of which is a PDE5 inhibitor or a PDE5 inhibitor analog. The molecular constituents may be two or more neutral molecules, two or more salts, or a complex containing one or more neutral molecules and one or more salts. Co-crystals may be prepared by melt crystallization, recrystallization from solvents, or by physically mixing the components together, for example, by grinding. See, e.g., O. Almarsson and M. J. Zaworotko, Chem. Commun., 17:1889-1896 (2004). Examples of multi-component complexes are well known in the art. See, for example, J. K. Haleblian, J. Pharm. Sci. 64(8):1269-88 (1975).

PDE5 inhibitors and PDE5 inhibitor analogs may have one or more chiral centers and thus exist as one or more stereoisomers. Such stereoisomer-containing compounds can be prepared and/or isolated as a single enantiomer, a mixture of enantiomers, a mixture of diastereomers, or a racemic mixture. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981). PDE5 inhibitors or PDE5 inhibitor analogs can be incorporated into compositions as a single enantiomer, a mixture of enantiomers, a mixture of diastereomers, or a racemic mixture of enantiomers.

B. Formulations

Pharmaceutical compositions including an agent that increases the level of cGMP in the intestinal mucosa or increases the activity of a PKG, preferably PKG2, in the intestinal mucosa are provided. The pharmaceutical compositions may be for administration by oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in unit dosage forms appropriate for each route of administration. In some embodiments, the composition is administered locally, to the site in need of therapy. For example, for treatment of cancer, the local administration includes administrative near the tumor or directly into the tumor (i.e., intratumoral).

1. Formulations for Enteral Administration

In a preferred embodiment the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

The agent that increases the level of cGMP in the intestinal mucosa or increases the activity of a PKG, preferably PKG2, in the intestinal mucosa may be chemically modified so that oral delivery of the derivative is efficacious. Generally, the chemical modification contemplated is the attachment of at least one moiety to the component molecule itself, where the moiety permits uptake into the blood stream from the stomach or intestine, or uptake directly into the intestinal mucosa. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. An agent that increases the level of cGMP in the intestinal mucosa or increases the activity of a PKG, preferably PKG2, in the intestinal mucosa can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e. the drug is enclosed in a semipermeable membrane which allows water to enter and push drug out through a single small opening due to osmotic effects.

For oral formulations, the location of release may be the stomach, the small intestine (the duodenum, the jejunem, or the ileum), or the large intestine. Preferably, the release will avoid the deleterious effects of the stomach environment, either by protection of the agent (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

2. Topical or Mucosal Delivery Formulations

Compositions can be applied topically. The compositions can be delivered to the lungs while inhaling and traverses across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent™ nebulizer (Mallinckrodt Inc., St. Louis, Mo.); the Acorn II™ nebulizer (Marquest Medical Products, Englewood, Colo.); the Ventolin™ metered dose inhaler (Glaxo Inc., Research Triangle Park, N.C.); and the Spinhaler™ powder inhaler (Fisons Corp., Bedford, Mass.).

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers.

3. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of disclosed compounds, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release 5:13-22 (1987); Mathiowitz, et al., Reactive Polymers 6:275-283 (1987); and Mathiowitz, et al., J. Appl. Polymer Sci. 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

4. Dosages and Effective Amounts

For all of the disclosed compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 100 mg/kg of body weight daily are administered to mammals. Generally, for intravenous injection or infusion, dosage may be lower.

The compositions disclosed herein are typical provided in an effective amount to reduce or alleviate one or more symptoms of a disease or disorder to be treated. Typically, the compositions disclosed herein are administered to a subject in need thereof in an effective amount to reduce or alleviate one or more symptoms of an intestinal cancer or an inflammatory bowel disease compared to control, for example, a subject that is not treated with the composition.

In some embodiments, the compositions are administered in an amount effective to induce a pharmacological, physiological, or molecular effect compared to a control that is not administered the composition. For example, as discussed in more detail below, the composition may be administered in an effective amount to, reduce proliferation, reduce apoptosis, induce downstream effectors of PKG mediated signaling, increase DUSP8/10 (MKP5) expression or activation, decrease JNK expression or activation, decrease Sox9 protein levels, inhibit Sox9 repressive activity, increase activity of an antioxidant, increase expression of one or more genes encoding an antioxidant include, but not limited to, MnSOD, PRDX3, Cat, GSTa3, GSR, GPX1, and GPX2, reduce redox stress and in cells compared to a control, and combinations thereof. The composition may also be administered in an effective amount to reduce proliferation at the base of intestinal crypts, reduce apoptosis at the luminal surface, increase differentiation of or numbers of secretory cells including, but not limited to, goblet cells and enteroendocrine cells in the gastrointestinal tract of a patient in need thereof.

As discussed above, a preferred composition for increasing level of cGMP in the epithelial mucosa increasing the activity of a PKG, preferably PKG2, in the intestinal mucosa includes one or more phosphodiesterase 5 inhibitors. A number of phosphodiesterase 5 inhibitors are commercially available in FDA approved dosages. Therefore, in some embodiments, the composition includes a commercially available phosphodiesterase 5 inhibitor in a FDA approved dosage. Examples of approved dosages include, but are not limited to those outlined in Table 1 below:

TABLE 1
Overview of FDA approved PDE 5 inhibitors

| | Active | | | | Solubility**² | |
|---|---|---|---|---|---|---|
| Brand Name | Ingredient | Manufacturer | Dose (mg)*² | IC₅₀ nM¹ | (mg/ml) | t_{1/2} [symbol] h¹ |
| Viagra | Sildenafil | Pfizer | 25, 50, 100 | 3.9 | 3.5 | 3-5 |
| Levitra | Vardenafil | Bayer | 2.5, 5, 10, 20 | 0.1-0.7 | 0.11 | 4-5 |
| Cialis | Tadalafil | Eli Lilly | 2.5, 5, 10, 20 | 0.94 | Insoluble | 17.5 |

*Human Dose
**Solubility in water
¹ [symbol] M, Kovar A, [symbol] B. The clinical [symbol]
² [symbol] [symbol]
indicates data missing or illegible when filed

C. Targeting to the Gastrointestinal Mucosa

In some embodiments, the disclosed compositions are targeted to the intestinal mucosa. In preferred embodiments, the compositions are targeted to the intestinal mucosa, and exhibit reduced targeting to, or absorption by smooth muscle cells. In some embodiments, oral dosage forms are enterically coated to control release of the active agent at a desired site along the intestinal tract. For example, the composition may be designed to be preferentially absorbed in the small intestine, the large intestine, or combinations thereof. Methods and agents for enteric coating are known in the art and can be determined based on site of delivery and the disease or disorder to be treated.

In some embodiments, the composition is modified to include one or more adhesive molecules, targeting ligands, or other targeting moieties, to facilitate targeting to the intestinal mucosa. The modifications may be made to the active agent itself, or a coating or encapulator such PEG. Targeting ligands of receptors expressed on the surface of the intestinal mucosa are known in the art. For example, the compositions disclosed herein can be targeted to the intestinal mucosa by attaching an aminopeptidase N (APN) specific targeting molecule such as those disclosed in U.S. Published Application No. 2001/0129525. Aminopeptidase N (APN) is a type II membrane glycoprotein, belonging to a family of membrane-bound metalloproteases. It is expressed in a variety of tissues including intestinal brush border membranes. Ligands that induce clathrin mediated internalization and transcytosis of the APN receptor include F4 fimbriae of enterotoxigenic Escherichia coli (ETEC). Therefore, in some embodiments, an F4 fimbriae, or fragment or variant thereof is used to target the compositions disclosed herein to the intestinal mucosa.

D. Combination Therapy

The disclosed compositions can be administered alone or in combination with one or more additional therapeutic agents. For example, the disclosed compositions can also be combined with one or more traditional therapies for treating inflammatory bowel diseases. Examples include, but are not limited to aminosalicylates, corticosteroids, immune modifiers such as 6-mercaptopurine and azathioprine, anti-TNF agents such as infliximab, antibiotics, antidiarrheal agents, antispasmodics, and acid suppressants.

For compositions for treating cancer, a preferred additional therapeutic agent is a DNA methylase inhibitor. Representative DNA methylase inhibitors include, but are not limited to 5-azacytidine, 5-aza-2′-deoxytidine, 1-β-D-arabinfurnosyl-5-azacytosine, dihydro-5-azacytidine or combinations thereof.

The disclosed compositions can be administered with an antibody or antigen binding fragment thereof specific for a growth factor receptors or tumor specific antigens. Representative growth factors receptors include, but are not limited to, epidermal growth factor receptor (EGFR; HER1); c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor eceptor 4; nerve growth factor receptor (NGFR) (TrkA); BDNF receptor (TrkB); NT-3-receptor (TrkC); vascular endothelial growth factor receptor 1 (Flt1); vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Axl; RYK; DDR; and Tie.

Additional therapeutic agents include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumour agents. All of these drugs affect cell division or DNA synthesis and function in some way.

Representative chemotherapeutic agents include, but are not limited to alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (inclding camptothecins such as irinotecan and topotecan and derivatives of epipodophyllotoxins such as amsacrine, etoposide, etoposide phosphate, and teniposide). Additional cancer therapeutics include monoclonal antibodies (e.g., trastuzumab (HERCEPTIN®), cetuximab (ERBITUX®), rituximab (RITUXAN® or MABTHERA®), and bevacizumab (AVASTIN®), and tyrosine kinase inhibitors (e.g. imatinib mesylate (GLEEVEC® or GLIVEC®)), which directly targets a molecular abnormality in certain types of cancer (chronic myelogenous leukemia, gastrointestinal stromal tumors).

Other suitable therapeutics include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

### III. Methods of Treatment

The compositions disclosed herein are useful for treating one or more symptoms of an inflammatory bowel disease, for treating one or more symptoms of an intestinal cancer, and for increasing intestinal luminal integrity. In some embodiments, the compositions are used to treat a disease or disorder of the gastrointestinal tract. In some embodiments, the compositions are used to treat diseases and disorders of the large intestine, for example, the colon, or a sub-region thereof such as the ascending colon, the traverse colon, the descending colon, the sigmoid fixture, or combinations thereof. In certain embodiment the composition is only active in a specific region or sub-region of the gastrointestinal tract, for example, the small intestine or the large intestine. In some embodiments the composition is targeted to a specific region or sub-region of the gastrointestinal tract.

A. Intestinal and Bowel Disorders

It has been discovered that compositions that increase the level of cGMP in the intestinal mucosa or increase the activity of a PKG, preferably PKG2, in the intestinal mucosa, can be used to reduce or alleviate one or more symptoms of an intestinal or bowel disease or disorder. The disclosed compositions can be administered to a subject in need thereof in an effective amount to increase the level of cGMP in the intestinal mucosa or increase the activity of a PKG, preferably PKG2, in the intestinal mucosa, for example in intestinal epithelial cells. In a preferred embodiment, a phosphodiesterase inhibitor, for example a PDE5 inhibitor such as vardenafil, is administered to patient in need thereof in an amount effective to reduce one or more symptoms of the intestinal or bowel disease or disorder.

Intestinal and bowel disorders include inflammatory bowel disorders. Representative inflammatory bowel disorders include, but are not limited to inflammatory bowel diseases such as Crohn's disease, ulcerative colitis, necrotizing enterocolitis, collagenous colitis, lymphocytic colitis, ischaemic colitis, diversion colitis, Behçet's disease, and indeterminate colitis. Symptoms of inflammatory disorders include, but are not limited to, abdominal pain, vomiting, diarrhea, rectal bleeding, severe internal cramps/muscle spasms in the region of the pelvis, weight loss, arthritis, pyoderma gangrenosum, and primary sclerosing cholangitis. Intestinal and bowel disorders also include conditions such as irritable bowel syndrome (IBS) which is a functional bowel disorder characterized by chronic abdominal pain, discomfort, bloating, and alteration of bowel habits. Symptoms of patients suffering from IBS include, but are not limited to, abdominal pain or discomfort, frequent diarrhea or constipation, a change in bowel habits, urgency for bowel movements, a feeling of incomplete evacuation (tenesmus), bloating or abdominal distention, gastroesophageal reflux, symptoms relating to the genitourinary system, chronic fatigue syndrome, fibromyalgia, headache, and backache.

Other intestinal disorders that can be treated using the disclosed compositions include gastroenteritis, ileitis, appendicitis, and coeliac disease.

B. Cancer

It has been discovered that compositions that increase the level of cGMP in the intestinal mucosa or increase the activity of a PKG, preferably PKG2, in the intestinal mucosa, can be used to reduce or alleviate one or more symptoms of cancer. In a preferred embodiment, the compositions are used to treat an intestinal cancer. Intestinal cancers including, but not limited to, duodenal cancer, ileal cancer, jejunal cancer, small intestine cancer, and colon (colorectal) cancer. The disclosed compositions can be administered to a subject in need thereof in an effective amount to increase the level of cGMP in the intestinal mucosa or increase the activity of a PKG, preferably PKG2, in the intestinal mucosa, for example, in intestinal epithelial cells. In a preferred embodiment, a phosphodiesterase inhibitor, for example a PDE5 inhibitor such as vardenafil, is administered to patient in need thereof in an amount effective to reduce one or more symptoms of the intestinal cancer. For example, in some embodiments, treatment results in a reduced number of tumor cells or tumor burden. In some embodiments the number of tumor cells or tumor burden is not increased.

C. Improving Intestinal Luminal Integrity

It has been discovered that PKG activity, particularly PKG2 activity participates in intestinal homeostasis. Therefore, compositions that increase the level of cGMP in the intestinal mucosa or increase the activity of a PKG, preferably PKG2, can be used to reduce or alleviate one or more pathologies of the intestinal mucosa.

The intestinal epithelium provides a barrier between antigenic luminal contents and the underlying immune cells. As used herein, “intestinal luminal integrity,” refers to the ability of the intestinal luminal surface to prevent or inhibit antigens in the lumen of the gastrointestinal tract from penetrating the luminal surface and accessing the underling immune cells. Intestinal mucosa with increased or improved luminal integrity is able to withstand more or greater insults, or exhibit greater protection from luminal antigens. Luminal integrity can include, but it not limited to, an intact mucosa at the luminal surface and a protective mucus barrier that separates the epithelial surface from luminal contents. It has been discovered that PKG activity contributes to luminal integrity of the intestinal mucosa. Therefore, compositions that increase the level of cGMP in the intestinal mucosa or increase the activity of a PKG, preferably PKG2, in the intestinal mucosa increase or improve luminal integrity. In some embodiments, the luminal surface of the intestinal mucosa of a subject who has been administered the composition is able to withstand more or greater insults, exhibit greater protection from luminal antigens, or combinations thereof.

In some embodiments, the composition increases or enhances intestinal luminal integrity by increasing or improving one or more physiological parameters of luminal integrity. For example, as discussed in detail below, activation of PKG2 in the colon can protect the luminal surface by reducing reactivity and apoptosis of luminal epithelial cells in response to inflammatory stimuli, enhancing the mucus layer by promoting increased differentiation or numbers of goblet cells, increasing stimulation of mucus synthesis and/or secretion by goblet cells, or combinations thereof.

Proliferation at the intestinal crypt base is tightly coupled to apoptosis at the luminal surface in order to maintain an effective barrier. The increased apoptosis observed in PKG2 knockout mice indicates that PKG2 does not block proliferation in the colon mucosa as suggested previously, but instead protects the surface from being killed. Loss of either GCC or PKG2 would then lead to increased epithelial apoptosis and compensatory proliferation in the crypt as the mucosa renews itself in an attempt to maintain a good barrier. In some embodiments a composition that increases the level of cGMP in the intestinal mucosa or increases the activity of a PKG, preferably PKG2, in the intestinal mucosa, is administered in an effective amount to reduce cell death (i.e., apoptosis) at the luminal surface. In some embodiments a composition that increases the level of cGMP in the intestinal mucosa or increases the activity of a PKG, preferably PKG2, in the intestinal mucosa, is administered in an effective amount to reduce proliferation or hyperplasia in the crypt base.

In some embodiments, a composition that increases the level of cGMP in the intestinal mucosa or increases the activity of a PKG, preferably PKG2, in the intestinal mucosa protects the epithelial surface from damaging and inflammatory luminal contents by inducing downstream effectors of PKG mediated signaling. For example, in some embodiments, a composition that increases the level of cGMP in the intestinal mucosa or increases the activity of a PKG, preferably PKG2, in the intestinal mucosa is administered in an effective amount to maintain, increase, or activate DUSP8/10 (MKP5) expression. In some embodiments, the composition is administered in an effective amount to maintain, reduce, or decrease JNK activity levels.

It is believed that differentiation of intestinal cells, particularly differentiated secretory lineage cells, are important for maintaining the luminal barrier. Examples of differentiated secretory cells include, but are not limited to, goblet cells and enteroendocrine cells. Therefore, in some embodiments a composition that increases the level of cGMP in the intestinal mucosa or increases the activity of a PKG, preferably PKG2, in the intestinal mucosa, is administered in an effective amount to increase the number of goblet cells or enteroendocrine cells. In some embodiments, the composition is administered in amount effective to increase differentiation of goblet cells or enteroendocrine cells. Sox9 regulation by PKG2 has an important role in maintaining intestinal homeostasis. For example, Sox9 regulation is believed to be important for promoting goblet cell differentiation. It has been discovered that PGK2 results in decreased Sox9 protein levels or inhibition of Sox9 repressive activity, or a combination thereof. Therefore, in some embodiments a composition that increases the level of cGMP in the intestinal mucosa or increases the activity of a PKG, preferably PKG2, in the intestinal mucosa is administered in an effective amount to decrease Sox9 protein levels or inhibit Sox9 repressive activity or a combination thereof.

Reactive oxygen species (ROS) and reactive metabolic intermediates generated from carcinogens or released from inflammatory cells are known to play an important role in cell damage and in the initiation and progression of carcinogenesis and inflammation. It has been discovered that compositions that increase the level of cGMP in the intestinal mucosa or increase the activity of a PKG in the intestinal mucosa can induces antioxidant activity. For example, in some embodiments, the composition increases activity of an antioxidant. In some embodiments, the composition increases expression of one or more genes encoding an antioxidant. Genes encoding antioxidants include, but are not limited to, MnSOD, PRDX3, Cat, GSTa3, GSR, GPX1, and GPX2. In some embodiments the composition reduces or alleviates stress induced by reduction-oxidation (redox) stress. For examples, the composition may reduce levels of dihydrodichlorofluorescein diacetate (DCF) fluorescence in cells compared to a control.

### IV. Methods of Detection

One embodiment provides a method for detecting or assisting in the diagnosis of an intestinal disorder, inflammatory bowel disease, intestinal cancer, or compromised luminal integrity, by obtaining a sample from a subject and assaying the sample for expression levels or activity of PKG2. Suitable samples include tissue or cells, preferably intestinal tissue or cells. Decreased expression of PKG2 in the sample relative to a control is indicative of reduced liminal integrity in the gastrointestinal tract. Methods for detecting the expression and activity of PKG2 are known in the art.

### V. Method of Screening for Compounds that Improve Luminal Integrity

Modulators of improved luminal integrity can be identified using the assays and reagents described herein as well as known techniques and reagents. Preferably the modulator increases the level of cGMP in the intestinal mucosa or increases the activity of a PKG, preferably PKG2, in the intestinal mucosa.

In some embodiments, screening assays can include random screening of large libraries of test compounds. Alternatively, the assays may be used to focus on particular classes of compounds suspected of modulating the level of cGMP in the intestinal mucosa or the activity of a PKG, preferably PKG2, in the intestinal mucosa. Assays can include determinations of PKG2 expression, protein expression or protein activity. Other assays can include determinations of PKG2 nucleic acid transcription or translation, for example mRNA levels, miRNA levels, mRNA stability, mRNA degradation, transcription rates, and translation rates.

In one embodiment, an assay for identifying modulators of intestinal luminal integrity is based on the level of cGMP in the intestinal mucosa or the activity of a PKG, preferably PKG2, in the intestinal mucosa in the presence and absence of a test compound. The test compound or modulator can be any substance that increases or is believed to increase the level of cGMP in the intestinal mucosa or increase the activity of a PKG, preferably PKG2, in the intestinal mucosa in a cell.

Specific assay endpoints or interactions that may be measured in the disclosed embodiments include increases the level of cGMP in the intestinal mucosa or increases the activity of a PKG, preferably PKG2, in the intestinal mucosa. These assay endpoints may be assayed using standard methods such as FACS, FACE, ELISA, Northern blotting, Western blotting, or combinations thereof. Moreover, the assays can be conducted in cell free systems, in isolated cells, genetically engineered cells, immortalized cells, or in organisms such as C. elegans and transgenic animals.

Another embodiment provides a method for identifying a modulator of the level of cGMP in the intestinal mucosa or the activity of a PKG, preferably PKG2, in the intestinal mucosa, by determining the effect a test compound has on luminal integrity in cells, tissue explants or in vivo. For example cells, tissue explants or whole organisms can be contacted with a test compound. Following administrative of the test compound, the cells, explant or tissue harvested from the whole organism can be assayed for reduced proliferation, reduced apoptosis, induction of downstream effectors of PKG mediated signaling, increased DUSP8/10 (MKP5) expression or activation, decreased JNK expression or activation, decreased Sox9 protein levels, inhibition of Sox9 repressive activity, increased activity of an antioxidant, increased expression of one or more genes encoding an antioxidant include, but not limited to, MnSOD, PRDX3, Cat, GSTa3, GSR, GPX1, GPX2, or reduce redox stress in cells compared to a control. When the test compound is administered to a tissue explant or in vivo, the tissue explant or tissue harvested from the whole organism can also be assayed according to histo-pathological phenotypes including by not limited to reduced proliferation at the base of intestinal crypts, reduced apoptosis at the luminal surface, and increased differentiation, numbers of secretory cells including, but not limited to, goblet cells and enteroendocrine cells.

Suitable cells for this assay include, but are not limited to, immortalized cell lines, and primary cell culture. In a preferred embodiment, the cells are colon cells, or colon cancer cells. Suitable colon cells lines are known in the art and described in the Examples below.

One example of an in vivo assay for testing the effectiveness of a test compound to improve intestinal luminal integrity is a murine model of inflammatory bowel disease. A well characterized mouse model of inflammatory bowel disease involves administration of dextran sulfate-sodium (DSS) in the drinking water. DSS is toxic specifically to luminal epithelial cells of the distal colon. For example, a test compound can be administered to mice beginning up to two days prior to introducing 5% dextran sulphate sodium (DSS) into their drinking water. After 5 days the DSS is replaced with water. Incidence and severity of diarrhea and body weight are recorded daily and combined in an index of disease severity. Signs of improved luminal integrity may include reduced mortality, reduced symptoms of colitis including but not limited to weight loss or diarrhea, or combinations thereof.

### EXAMPLES

### Example 1

### PKG2 is Expressed in the Gastrointestinal Tract

The intestinal tract from duodenum to distal colon was removed from euthanized mice (blot shown is representative of at least 3 different animals), and the mucosa scraped from underlying connective tissue. Mucosal extracts were prepared for SDS-PAGE and Western blotting to detect PKG1 and PKG2. Beta-actin was detected as a loading control.

### Example 2

### PKG2 Suppresses Proliferation in the Colon

### Material and Methods

Animals

The type 2 PKG knockout mouse was provided by Dr Franz Hofmann (Technische Universität Munchen) (Pfeifer, et al., Science, 274:2082-6 (1996)) and was maintained with the GHSU Laboratory Animal Service Standard Operating Procedures. Genotyping PCR was performed using 1 μl genomic DNA (purified by Gentra Puregene

Mouse Tail kit, QIAGEN, Valencia, Calif.) with a common forward primer: AAGATAGTCCTATCAAATCATGGAACG (upstream of neomycin. insert at the end of 1st exon), a primer to detect knockout:
CATAGCGTTGGCTACCCGTGATATTGC (within the neomycin insert) and reverse for wild type: GCCGCTACAGGACAGATAAGAGACGAA (intron sequence downstream of neomycin insert).

Statistical Analysis

All quantitative experiments were reproduced in at least three independent experiments with multiple wells in each replicate. The resulting data were expressed as means with error bars indicating SEM. Two means were considered to be statistically significant when two-tailed Student T-test had pvalue less than 0.05.

### Results

Loss of either guanylin or its effector GCC in IEC compromises cGMP signaling and results in increased crypt size due to expansion of the proliferative compartment (Steinbrecher, et al., Am J Pathol, 161-2169-78 (2002) and Li P, et al., Am J Pathol, 171:1847-58 (2007)). PKG2 is an important effector of cGMP in the intestinal mucosa but its role in homeostasis downstream of guanylin/GCC has not been investigated. A Prkg2−/− mouse that was created previously to demonstrate the function of PKG2 in intestinal secretion (Pfeifer, et al., Science, 274:2082-6 (1996)) was characterized as described herein to determine the role of PKG2 on intestinal homeostasis.

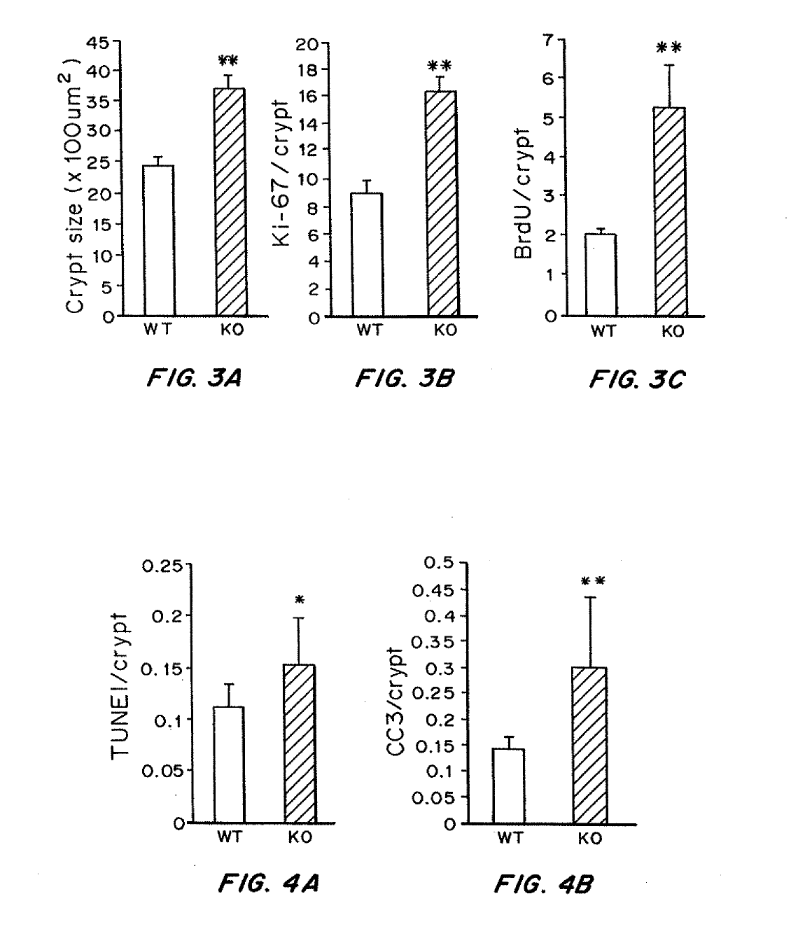

RT-PCR and immunoblotting confirmed the absence of PKG2 expression in the colon mucosa. Prkg2−/− animals have a smaller stature than wild types owing to defective endochondral ossification (Pfeifer, et al., Science, 274:2082-6 (1996)) but preliminary examination did not detect any notable macroscopic differences in the intestine. Histological examination also did not show obvious differences in the organization of the mucosa, but careful measurement revealed significantly longer crypts in the Prkg−/− relative to wild type animals (FIG. 3A). The increase in crypt length was most pronounced in the proximal colon with a smaller but significant effect in the ileum and distal colon (p<0.05, data not shown). There were significantly more proliferating cells in the crypts of Prkg2−/− animals relative to wild type siblings as detected with either Ki67 staining or BrdU incorporation (FIGS. 3B and 3C). The relative position of the proliferating cells corresponded to the transit amplifying region, indicating that the increased crypt length is due to expansion of the progenitor cells. There are conflicting reports regarding intestinal apoptosis in GCC knockout animals relative to wild type (Li P, et al., Am J Pathol, 171:1847-58 (2007), Li P., Gastroenterology, 133:599-607 (2007), Garin-Laflan M P, et al., Am J Physiol Gastrointest Liver Physiol, 296:G740-9 (2009) and Steinbrecher K A, et al., J Immunol, 186:7205-14 (2011)). Significantly more apoptotic cells were observed in the proximal colon epithelium of the Prkg2−/− animals compared to wild type siblings when measured by either TUNEL or cleaved-caspase 3 immunohistochemistry. The stained tissue samples were quantified and graphed (FIGS. 4A and 4B).

### Example 3

### PKG2 Promotes Differentiation of Secretory Lineage Cells in the Colon

### Materials and Methods

Immunohistochemistry and Immunofluorescence

Cells on coverslips were fixed with 3.7% formaldehyde. Alcian blue/periodic acid-Schiff (AB&PAS) stain for mucopolysaccharide was done with standard procedures in the GHSU core histology facility. Antibodies to MUC2 from Santa Cruz (1:200 dilution, Santa Cruz, Calif.), and DAPI from Calbiochem (San Diego, Calif., USA) for nuclear staining Alexa Fluor 568 secondary antibodies (Invitrogen, Carlsbad, Calif.) were used and VectorMount AQ mounting medium to fix slides. Antigens were unmasked in paraffin-embedded sections (7 μm) by heating at 100° C. for 30 min in 10 mM sodiumcitrate (with 0.05% Tween 20, pH=6). Antibodies to Ki-67 were from Dako Cytomation (1:50 dilution, Carpinteria, Calif.), to BrdU were from Abcam (1:100 dilution, Cambridge, Mass.), to MUC2 were from Santa Cruz (1:200 dilution, Santa Cruz, Calif.), and to Chromogranin A were from Immunostar (1:200 dilution, Hudson, Wis.). Alkaline phosphatase was stained with the Alkaline Phosphatase kit I (catalog No. SK-5100, Vector, Burlingame, Calif.). Analysis was done independently by two individuals who were blinded to the mouse genotypes, and three mice of each genotype were analyzed.

### Results

The increased crypt size and hyperplasia measured in the proximal colon of Prkg2−/− mice has also been observed in animals deficient in guanylin (Gn) (Steinbrecher K A, et al., Am J Pathol, 161:2169-78 (2002)) and its receptor, GCC (Li P, et al., Am J Pathol, 171:1847-58 (2007)). The GCC-deficient mice were also reported to have reduced numbers of goblet cells in the colon with no difference in enteroendocrine cells.

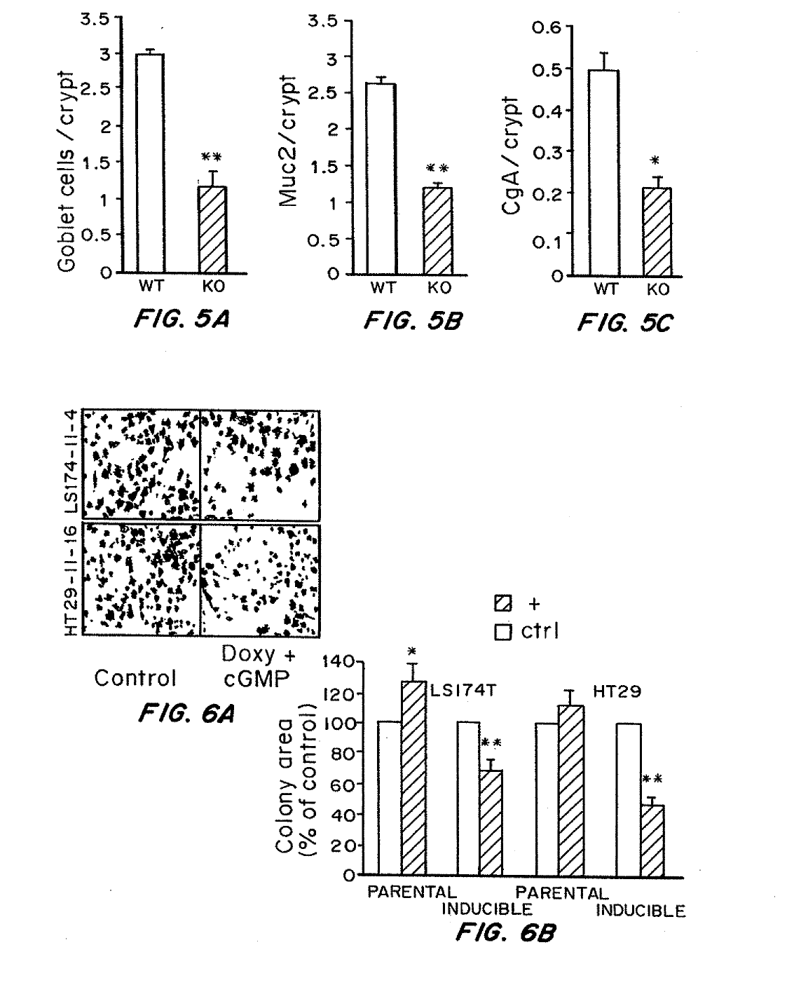

To determine whether PKG2 influences differentiation of secretory cells, sections of proximal colons from Prkg2−/− and wild type animals were stained for goblet cells and enteroendocrine cells and then quantified (FIG. 5A). Staining with either alcian blue/periodic acid Schiff (AB/PAS) or anti-Muc2 antibodies revealed numerous oligomucous cells deeper in the crypts without notable difference between Prkg2−/− and wild type animals. In contrast, the Prkg2−/− animals had significantly less mature goblet cells in the upper regions of the crypts than wild type animals (approximately ⅓rd, p<0.001). Very similar observations were obtained independent of whether AB/PAS or Muc2 staining was used (FIGS. 5B and 5C).

Enteroendocrine cells represent the other secretory lineage cell in the colon that was quantitated in colon sections by staining for chromogranin A. Much like the goblet cells, there were significantly fewer enteroendocrine cells in the proximal colons of Prkg2−/− animals relative to wild type siblings (approximately ½, p<0.05). Staining for alkaline phosphatase showed no difference in colonocyte numbers between Prkg2−/− and wild type animals (data not shown) suggesting that PKG2 is necessary for the differentiation or maintenance of secretory cells in the colon.

The colons of Prkg2−/− animals exhibited an exaggerated proliferative compartment and a larger crypt size relative to Prkg2+/+ siblings but there was no evidence of dysplasia or hyperplasia outside of the crypts. This effect was observed in both ileum and distal colon but was most pronounced in the proximal colon (Example 2). In addition to the increased proliferation, the Prkg2−/− animals showed significantly higher numbers of apoptotic cells and reduced numbers of secretory goblet and enteroendocrine cells (Examples 2 and 3). The intestinal phenotype of the Prkg2−/− mice is remarkably similar to the GCC knockout animals (Ramakrishna B S, Trop Gastroenterol, 30:76-85 (2009)) and strongly indicates that PKG2 mediates the homeostatic effects of GCC/cGMP in the colon. The reduced effect of PKG2 deficiency in the distal colons likely reflects the expression pattern of PKG2, which decreases along a rostrocaudal axis and is virtually undetectable in the distal colon (Markert T, et al., J Clin Invest, 96:822-30 (1995)). Consistent with the importance of PKG2 downstream of cGMP, both guanylin (Steinbrecher K A, et al., Am J Pathol, 161:2169-78 (2002)) and GCC (Ramakrishna B S, Trop Gastroenterol, 30:76-85 (2009)) knockout animals also showed less pronounced homeostatic effects in the distal colon.

The striking reduction in goblet and enteroendocrine cells in the Prkg2−/− colons in the absence of obvious changes in the numbers of absorptive colonocytes suggests an important role for PKG2 in either secretory lineage specification or in the terminal differentiation of these cells. Reduced numbers of goblet cells has also been reported in GCC knockout mice but that study did not observe significant changes in enteroendocrine cells (Ramakrishna B S, Trop Gastroenterol, 30:76-85 (2009)). The results described herein indicate that PKG2 mediates the differentiation promoting functions of GCC signaling. Goblet cells produce and secrete a protective mucus barrier that separates the epithelial surface from luminal contents, and loss of this layer results in spontaneous colitis and tumorigenesis (Johansson M E V, et al., National Academy of Sciences, 105:15064-15069 (2008), Van der Sluis M, et al., Gastroenterology, 131:117-129 (2006), Mizoshita T, et al., Histol Histopathol, 22:251-60 (2007) and Velcich A, et al., Science, 295:1726-9 (2002)). Muc2−/− mice also exhibit an exasperated inflammatory response to colonocyte toxins such as DSS (Van der Sluis M, et al., Gastroenterology, 131:117-129 (2006)).

### Example 4

### DSS Responsiveness in Prkg2−/− is Similar to Wild Type

### Materials and Methods

Treatment and Pathological Grading of Inflammatory Colitis The Dextran Sodium sulfate (DSS) model of colonic wounding was performed as previously detailed 23. In brief, 4% DSS (m.w. 36,000-50,000; MP Biomedicals) in drinking water was provided to 8 week-old male mice for 6 days followed by water for 8 days for recovery. Intestinal tissues were harvested as swiss rolls immediately on sacrifice of animals and processed for formaldehyde fixation and paraffin embedding. Disease activity index included a summation of three components: weight loss (0=0%, 1=1-5%, 2=5-10%, 3=10-20%, 4=>20%), diarrhea (0=normal stool, 1=stool soft but formed, 2=stool very soft, 3=diarrhea, 4=dysenteric diarrhea), and rectal bleeding (0=abscent, 1=occult bleeding, 2=blood around anus, 3=blood on fur/tail, 4=gross macroscopic bleeding).

### Results

Goblet cells have the important role of secreting a protective mucous layer that separates the IEC from commensal bacteria (Johansson M E V, et al., National Academy of Sciences, 105:15064-15069 (2008)). Muc2 is the main protein component of intestinal mucous and Muc2−/− mice exhibit a high basal level of colitis (Van der Sluis M, et al., Gastroenterology, 131:117-129 (2006)). The defective mucous barrier in Muc2−/− mice also makes them particularly sensitive to dextran-sulfate sodium (DSS) induced experimental colitis (Van der Sluis M, et al., Gastroenterology, 131:117-129 (2006) and Johansson M E, et al., PLos One, 5:e12238 (2010)).

DSS responsiveness in Prkg2−/− relative to wild type animals was examined. Wounding of the colon by including 4% DSS in the drinking water for 5 days caused a decrease in body weight of both wild type and Prkg2−/− animals without significant difference between the two. The disease activity index (weight change, bleeding, and diarrhea) and histological evaluation also did not demonstrate a significant effect of PKG2 status on sensitivity to DSS treatment.

### Example 5

### PKG2 Inhibits the Proliferation of Colon Cancer Cell Lines

### Materials and Methods

Tissue Culture and Reagents

All cell lines were obtained from the American Type Culture Collection (ATCC) and maintained in 5% CO2 in RMPI-1640 medium containing 10% FBS, and supplemented with 200 μM L-glutamine, 10 IU/ml penicillin, 10 mg/ml streptomycin. Cell lines made inducible for type 2 PKG expression in response to doxycycline (doxy) were generated using the Lenti-X™ lentiviral inducible expression system from Clonetech (Mountain View, Calif., USA)). The medium used to maintain stocks of inducible cells was supplemented with 200 μg/ml of G418 and 0.2 μg/ml of puromycin. Before experimentation, the cells were grown up for at least one passage in the absence of antibiotics. The doxy, DAPI and 8BrcGMP were form Calbiochem (San Diego, Calif., USA). NP-40, tween-20, puromycin were from Sigma (St Louis, Mo.). G418 was from Hyclone (Logan, Utah), and all other chemicals were from Fisher Scientific (Pittsburgh, Pa.).

Western Blotting

Cells were lysed by incubation in ice cold lysis buffer (50 mM HEPES pH 8.0, 150 mM NaCl, 1% Nonidet P-40, 0.25% deoxycholate) supplemented with protease inhibitor cocktail (Calbiochem, La Jolla, Calif.). Lysates were subjected to immunoblot analysis using antibodies to the following antigens: PKG2 from Santa Cruz (1:200 dilution, Santa Cruz, Calif.), Sox9 from Abcam (1:200 dilution, Cambridge, Mass.), Flag (1:1000) and β-actin (1:2000) antibodies from Sigma (St. Louis, Mo.), phosphorylate AKT at serine 473 and AKT from Cell Signaling (both 1:1000 dilution, Beverly, Mass.).

Colon cancer cell lines SW480, SW620, LS174T, KM12L4, HT29, HCT116, Colo201, and Colo205 and HT29 cells transfected with a PKG2 expression vector (positive control) were examined by immunoblot. Beta-actin was used as a loading control.

Immunoblots for PKG2 in lysates from cell lines LS174-II-4, HT29-II-16, and SW480-II-11 engineered to express PKG2 in the presence of doxycycline, with (+) and without (−) doxycycline +8Br-cGMP (Doxy+cGMP) were also performed. Functional PKG2 is shown as a mobility shift of the PKG target vasodilator-stimulated phosphoprotein (VASP) to phosphorylated VASP (VASP-P). Beta-actin is a loading control.

Analysis of Gene Expression

Steady state RNA levels were measured by semiquantitative RT-PCR. The cDNA was generated using GeneAmp PCR kits (Applied Biosystems, Foster City, Calif.). Gene-specific primers were designed using the National Center for Biotechnology Information (NCBI) Primer Blast Software as follows: human PKG2 (forward: AGCCCGCTTCAGGCCTCT CC, reverse: AGTCGACCCTCTGCCAGCAC), human Sox9 (forward: GATCTGAAGAAGGA GAGCGAGGAGGACAAG, reverse: CGTTGGGGGAGATGTGCGTCTGCTC), human MUC2 (forward: CCGCTGGAGCCGTATCTGCG, reverse: CGGGGCAGGGCAGGTCTTTG), human CDX2 (forward: AGGCAGAAGAGCCGCGAGGA, reverse: CCAGTCCTCCCGGAGTGGG G), mouse PKG2 (forward: GTGGCGGAGGACGCCAAGAC, reverse: AGGCCTGCAGTGG GCTTCCT), and common HPRT1 (forward: GCGTCGTGATTAGCGATGATGAAC, reverse: CCTCCCATCTCCTTCATGACATCT). Luciferase reporter assays were performed as described previously (Kwon I K, et al., Oncogene, 3423-34. The Flag-Sox9, MUC2- and CDX2-luciferase reporter constructs construct were described previously (Blanche P, et al., J Cell Biol, 166:37-47 (2004)).

Colon cancer cell lines SW480, SW620, LS174T, KM12L4, HT29, HCT116, Colo201, and Colo205 and human colon cancer tissue were examined. HPRT1 was used as a loading control.

RT-PCR of RNA isolated from cell lines LS174-II-4, HT29-II-16, and SW480-II-11 engineered to express PKG2 in the presence of doxycycline, with (+) and without (−) doxycycline (Doxy) were performed. HPRT1 was used as a loading control.

Cell Viability Determination

For colony formation assays the cells were seeded in 6-well plates at a density of 5000 cells/well in the presence or absence of PKG2 inducer doxy (2 μg/ml) and 8Br-cGMP (100 μM). Media with fresh doxy/8Br-cGMP was changed every other day and colonies formed after 14 days were fixed in 100% methanol for 30 min and stained with KaryoMax Gimsa stain (Sigma, St. Louis, Mo.) for 1 hr. Stained dishes were scanned at high resolution and the total colony area for each well was determined using Scion Image software (Scion Corporation, Frederick, Md.). For direct cell counts, the cells were seeded in six-well dishes with 400,000 cells/well and in the presence or absence of doxy/8Br-cGMP for 72 hours. Cells were detached and resuspended in PBS containing 0.2% trypan blue, and after 5 min the number of living (unstained) and dead (stained with blue) cells in each well were counted by in a hemocytometer. MTT cell proliferation assays were also used to measure cell growth. For these studies the cells were cultured in 96 well plates (5000 cells/well) in the presence or absence of Dox/8Br-cGMP, and after 48 h the MTT assays were performed according to the manufacturer's instructions (ATCC, Manasses, Va.).

Cells were seeded in 12-well plates at 50% confluence and either untreated or treated with 2 μg/ml doxy and 100 μM 8Br-cGMP. 48 hrs later, cells were washed twice in cold 1×PBS, harvested using trypsin-EDTA solution and cell death was assessed using annexin-V fluorescein isothiocyanate (FITC) and propidium iodide double staining according to the manufacturer instructions (Annexin-V FITC Apoptosis Kit, BD Bioscience, CA).

### Results

To better understand PKG2 signaling in the colon, its expression in colon tumors and cell lines was examined by RT-PCR and Western blotting. PKG2 was abundant in both normal and tumor tissue from the colon, but was undetectable in all colon cancer cells lines examined. This pattern of expression is reminiscent of upstream GCC expression, which has been highlighted as a marker of metastases of intestinal origin but is absent from many colon cancer cell lines (Waldman, et al., Cancer Epidemol Biomarkers Prey, 7:505-14 (1998)). Cell lines such as T84 and CaCo2 that retain GCC expression have been instrumental in characterizing downstream signaling. However, since PKG2 was not detected in any of the cell lines (including T84 (Markert T, et al., J Clin Invest 96:822-30 (1995)), several colon cancer cell lines were created that inducibly express PKG2 in response to doxycycline (doxy). Treatment of these cell lines with doxy resulted in robust increases in PKG2 mRNA and protein. Moreover, using mobility shift of the substrate vasodilator stimulated phosphoprotein (VASP) as readout, stimulation of doxy-treated cells with a membrane permeable analog of 8Br-cGMP demonstrated inducible PKG2 activity.

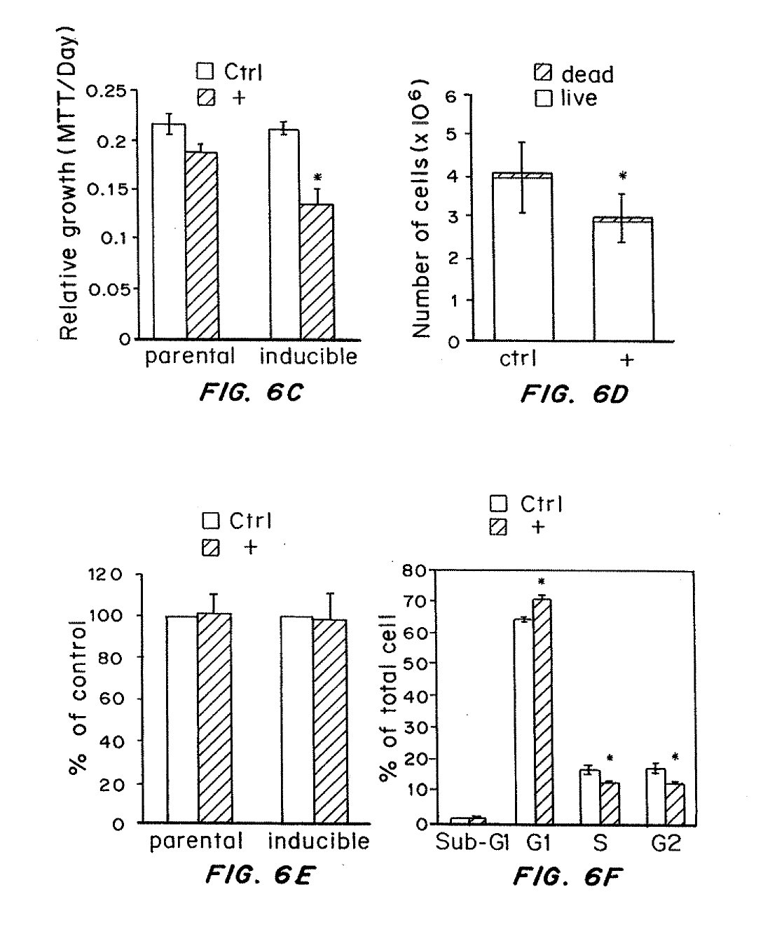

To test if inhibiting the proliferation of progenitor cells is a function of PKG2 in wild type animals, an in vitro assay was established. Colony formation assays were used to determine the effect of PKG2 expression on the proliferation of colon cancer cell lines. These studies showed that ectopic PKG2 inhibited colony formation in both LS174T (30%) and HT29 (50%) (FIGS. 6A and 6B). Under identical conditions of doxy and 8Br-cGMP, the parental cell lines that do not express PKG2 showed no significant effect (HT29) or slightly increased colony formation (LS174T). Similar effects of PKG2 on cell proliferation were obtained using MTT assays (35% decrease) and direct cell counting in a hemocytometer (25% decrease) (FIGS. 6C and 6D).

While ectopic PKG2 reduced proliferation of viable cells, it did not significantly affect trypan-blue positive cells. The ability of PKG2 to regulate cell death was further explored using annexin/propidium staining and cell cycle analysis, but neither approach was able to detect a significant effect (FIGS. 6E and 6F). While the cell cycle analysis did not measure an effect of PKG2 on sub-G1 cells, there was a significant increase in G1 cells (12.5%) and a decrease in the S+G2/M phase cells (25.6%). These results are similar to signaling downstream of GCC where cGMP-dependent cytostatic effects have been reported without any effect on apoptosis.

PKG2 expression is not reduced in colon tumors (not shown) but was not detected in any of the colon cancer cell lines examined. Ectopic re-expression of PKG2 inhibited the growth of both HT29 and LS174T cells without affecting cell death. This is reminiscent of the effects of GCC activation in colon cancer cells (Pitari G M, et al., Proc Natl Acad Sci USA, 100:2695-9 (2003) and Pitari G M, et al., Proc Natl Acad Sci U S A, 98:7846-51 (2001)) and supports the idea that PKG2 mediates anti-proliferation signaling downstream of GCC in the colonic crypts.

### Example 6

### PKG2 Promotes the Differentiation of Colon Cancer Cells

### Methods

Immunohistochemistry with AB & PAS staining, Muc2 immunofluorescence, and DAPI nuclear counterstaining in LS1747-II-4 cells with (PKG2) and without (Ctrl) Doxy+8Br-cGMP induction was performed.

Immunoblotting of PKG2 and the loading control beta-actin were also performed.

RT-PCR for Muc2, CDX2, and PKG2 in LS1747-II-4 cells with (PKG2) and without (Ctrl) Doxy+8Br-cGMP induction over time (hours) was performed. HPRT2 was used as a loading control.

### Results

The Prkg2−/− mice had less goblet and enteroendocrine cells in the proximal colon than wildtype animals. PKG2 has been reported to increase differentiation markers in glioma cell lines and in proliferating chondrocytes in the developing bone (Chikuda H, et al., Genes Dev, 18:2418-29 (2004) and Swartling F J, Oncogene, 28:3121-31 (2009)). To determine if PKG2 facilitates terminal differentiation in the colon mucosa, histological staining for polysaccharides and immunostaining for Muc2 was carried out as a measure of goblet cell differentiation. There was a relatively high background staining of mucopolysaccharides in the LS174T cells but this increased between 24-48 h after PKG2 induction and activation. In contrast, there was very little background staining in HT29 cells and this did not change when with ectopic PKG2.

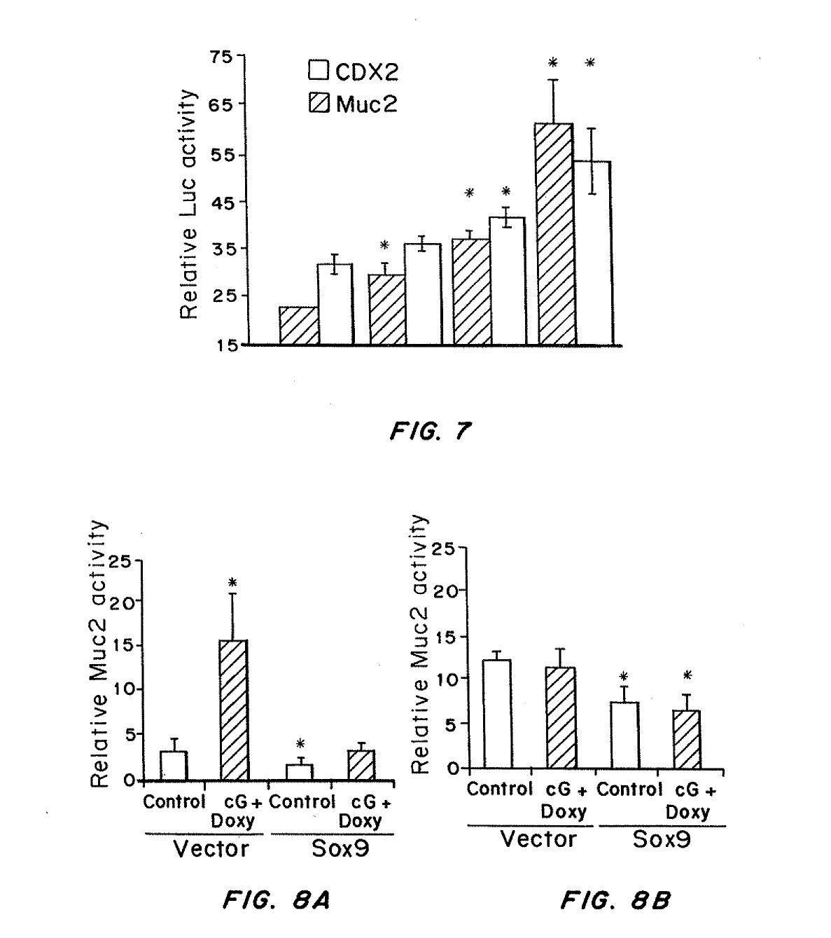

The regulation of Muc2 expression by PKG2 was examined further using RT-PCR, which showed that PKG2 activity resulted in increased Muc2 mRNA. CDX2 is a member of the Caudal homeobox genes with a critical role in the regulation of intestinal homeostasis (Coskin M., et al., Biochim Biophys Acta, 1812:283-9 (2011)). CDX2 activity is associated with differentiating IEC and is a direct transcriptional activator of Muc2 (Mesquita P, et al., J Biol Chem, 278:51549-56 (2003) and Yamamoto H, et al., Biochem Biophys Res Commun, 300:813-8 (2003)). Ectopic PKG2 also increased CDX2 mRNA levels in LS174T cells with similar kinetics to Muc2. Consistent with a transcriptional regulatory mechanism, PKG2 dose-dependently increased transcription from luciferase reporter constructs driven by upstream promoter regions from Muc2 (3 fold) and CDX2 (1.7 fold) genes (FIG. 7). In agreement with AB/PAS and Muc2 immunostaining, the increased expression of both CDX2 and Muc2 by activation of ectopic PKG2 was observed in LS174T cells but not in HT29 cells.

### Example 7

### Sox9 is a Downstream Effector of PKG2

The original report of Prkg2−/− mice described reduced GCC-mediated secretion in the intestine but it was noted that these animals also exhibited dwarfism owing to defective endochondral ossification (Pfeifer A, et al., Science, 274:2082-6 (1996)). Subsequent work by an independent laboratory demonstrated that PKG2 blocks Sox9 repressor activity leading to hypertrophic differentiation of chondrocytes (Chikuda H, et al., Genes Dev, 18:2418-29 (2004). As a target of the Wnt/beta-catenin pathway, Sox9 also has an important role in intestinal homeostasis and is a transcriptional repressor of Muc2 and CDX2 genes in colon cancer cell lines (Blance P., J Cell Biol, 166:37-47 (2004)).

Inhibition of Sox9 repressor activity by PKG2 was tested for a role in the differentiation of LS174T colon cancer cells. Over-expression of Sox9 reduced the basal level of transcription from the Muc2-luciferase reporter in LS174T and HT29 cells (FIGS. 8A and 8B). These results support the idea that partial differentiation of LS174T cells by PKG2 could result from inhibition of Sox9 repressive activity in these cells. PKG2 was reported to inhibit Sox9 in chondrocytes by blocking nuclear translocation (Chikuda H, et al., Genes Dev, 18:2418-29 (2004)), but this was not observed in the colon cancer cells examined here, where Sox9 was exclusively nuclear regardless of PKG2 status (data not shown). The nuclear localization of Sox9 was also not affected by PKG2 in glioma cells, but in that system PKG2 inhibited Sox9 expression at both protein and mRNA levels (Swartling F J, Oncogene, 28:3121-31 (2009)). Immunblots of Sox9, PKG2, and the loading control beta-actin from material isolated from LS174T-II-4 and HT29 cells treated as described in 8A and 8B were preformed. Overexpression and activation of PKG2 in LS174T cells resulted in decreased Sox9 protein, but not in HT29 cells, where a slight increase in Sox9 was observed. Additionally, while PKG2 reduced endogenous Sox9 levels, there was no effect on the level of transfected Sox9 driven by a constitutive CMV promoter. This indicated that post-translational mechanisms were probably not responsible for the change in Sox9 protein.

To explore this further, RT-PCR was used to show that expression and activation of PKG2 had no effect on Sox9 mRNA levels in HT29 cells, but markedly reduced the steady state level in LS174T cells. The RT-PCR of PKG2 and Sox9 was performed in LS174T-II-4 and HT29 with (+) and without (−) Doxy+8Br-cGMP induction of PKG2. HPRT1 was used as a loading control.

PKG2 was shown reduce the levels of Sox9 mRNA and protein in LS174T cells in association with increased CDX2 and Muc2, but not in HT29 cells where these differentiation markers were unaffected. These findings support the idea that PKG2 promotes secretory lineage-commitment or maturation by blocking Sox9 expression.

### Example 8

### PDE5 Inhibitors Increase cGMP Levels In Colon Cancer Cells

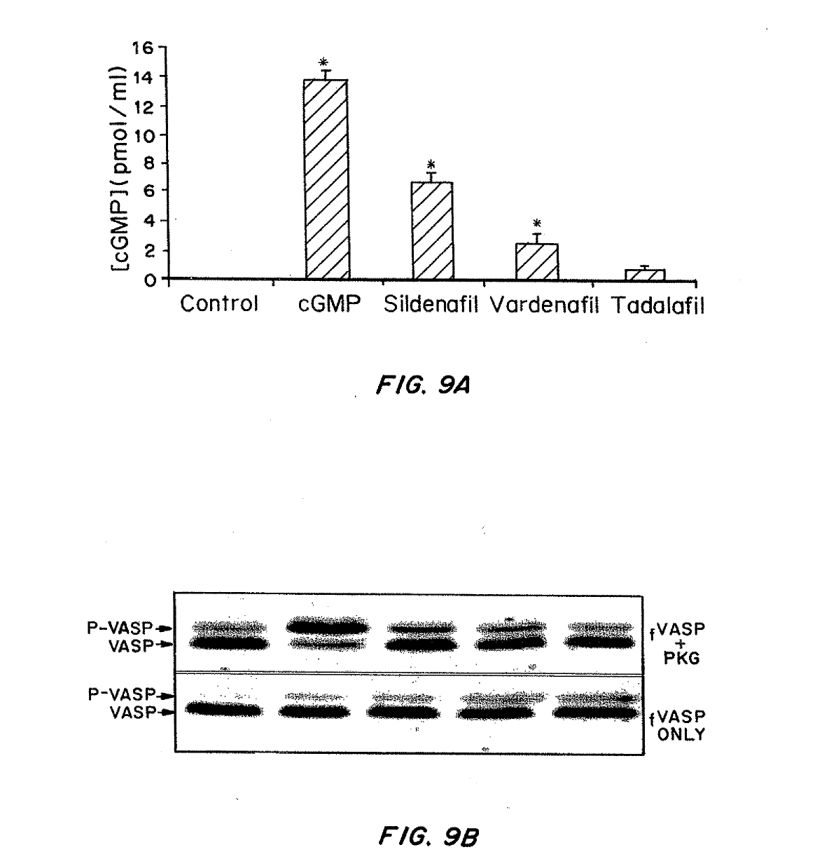

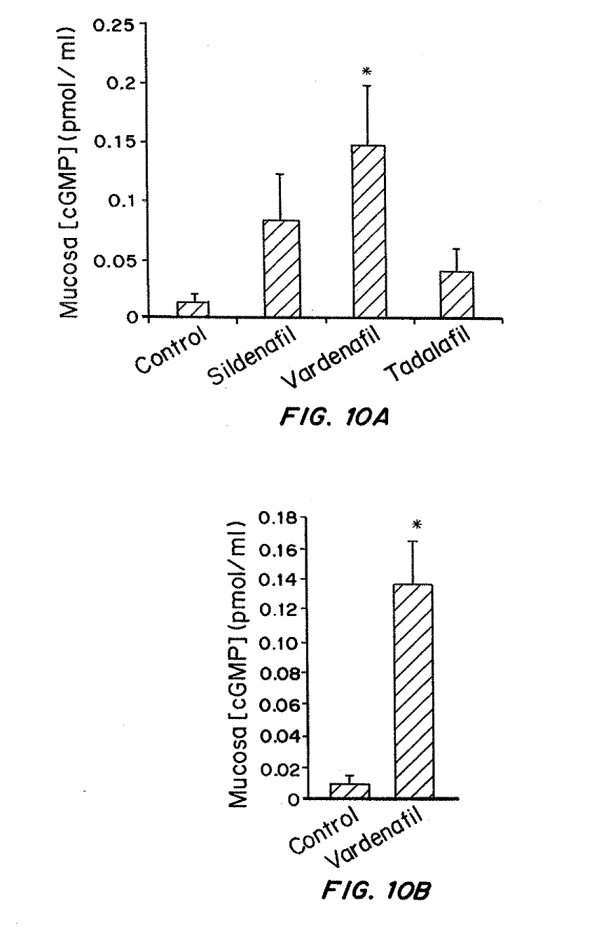

HCT116 colon cancer cells were incubated for 2 hours with PDE5 inhibitors (as indicated) and cell extracts were harvested for analysis of cGMP content using a competitive enzyme-linked immunosorbent assay (ELISA) (FIGS. 9A and 9B).

Activation of PKG caused increased phosphorylation of vasodilator-stimulated phosphoprotein (VASP) and reduced mobility on a western blot. Colon cancer cells were transfected with flag-tagged VASP (FIG. 9B both upper and lower panels) or VASP and PKG1 (FIG. 9B upper panel only). The cells were then incubated 2 hours with PDE5 inhibitors (as indicated) and harvested for immunoblot analysis for VASP mobility shirt using anti-flag antibodies. Results shown are representative of 3 experiments, and error bars indicate SEM.

PDE5 inhibitor treatment is sufficient to increase cGMP levels and activate PKG in colon cancer cell lines. The data in FIGS. 10A and 10B show that both Sildenafil and Vardenafil significantly increased intracellular cGMP levels in colon cancer cell lines. The increase in cGMP in the cancer cells corresponded with activation of PKG as measured by phosphorylation of its substrate vasodilator stimulated phosphoprotein (VASP).

### Example 9

### PDE5 Inhibitors are Effective in Mouse Colon Mucosa

CD1 mouse colon explants were incubated with PDE5 inhibitors in vitro. After 2 hours, the mucosa was harvested for analysis of cGMP content by ELISA (FIG. 10A).

Mice received intraperitoneal injection of vardenafil with 100 μl of olive oil as the vehicle. The mice were sacrificed 4 hours later and colon mucosa was harvested for analysis of cGMP content by ELISA. The cGMP levels were standardized for protein content using OD280 of the extracts. The error bars show SEM (FIG. 10B).

PDE5 inhibitor treatment is sufficient to increase cGMP levels in the colon mucosa both in vitro and when systemically administered to the whole animal. In mouse colon explants Vardenafil was able to significantly increase cGMP levels. Intraperitoneal injection of Vardenafil in vivo also resulted in a significant increase in intracellular cGMP levels in mouse colon mucosa (FIG. 10B). Intraperitoneal administration of vardenafil was able to partly mimic antioxidant effects of exogenous cGMP, indicating that PDE5 inhibitors may have therapeutic potential in diseases affecting the colon.

### Example 10

### Vardenafil Treatment Reduces Basal Apoptosis and Proliferation, and Increases Differentiation in the Colon Mucosa

### Materials and Methods

Mice were treated with 5 mg/kg Vardenafil for 7 days (IP/B.I.D.) and subsequently sacrificed and the colons processed for histochemistry. Sections were stained for apoptotic cells using TUNEL in control and treated. TUNEL positive cells were found. FIG. 12A shows the quantitation of cell death in the colon mucosa by TUNEL assay. Proliferation in the colon mucosa of control and Vardenafil treated animals was found by staining for incorporation of BrdU. FIG. 12B Quantitation of cell proliferation in the colon mucosa by BrdU incorporation. Asterisks in FIGS. 12A and 12B show statistical significance, p<0.05 of treated relative to control.

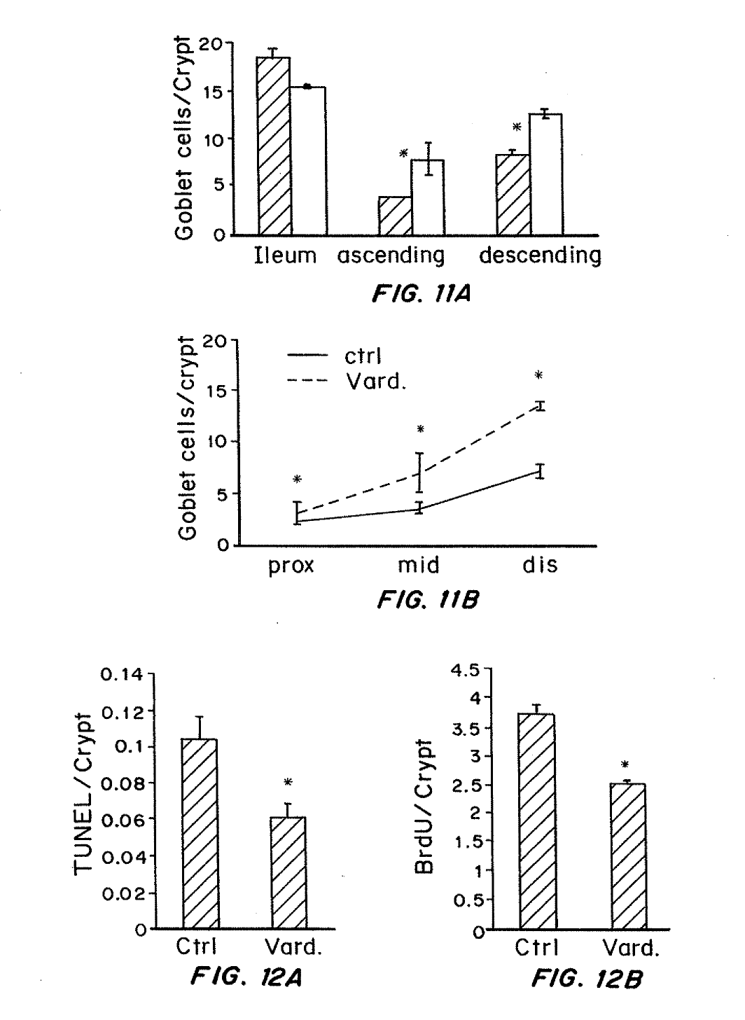

Mature goblet cells were counted in different sections of the colon following staining with Alcian blue-periodic acid schiff. FIG. 11A: Different regions of the gut (ilium of small intestine, ascending and descending portions of the colon). FIG. 11B: Relative mature goblet cell number in different sections of the proximal colon. Asterisks indicate significance (p<0.05, n=3).

AB+PAS staining of sections from the proximal, middle, and distal colon of control mice and mice treated with 5 mg/kg vardenafil (IP, b.i.d.) for 7 days were performed.

### Results

As shown in FIGS. 12A and 12B, the epithelial cell death and crypt proliferation were reduced in the colons of vardenafil treated animals. As shown in FIG. 11A-11B, the goblet cell numbers were significantly increased vardenafil treated animals. Treatment of mice with Vardenafil had the opposite effect on colon homeostasis as was observed in the PKG2 knockout animals and therefore strongly supports the idea that PKG2 has this important homeostatic role in the colon.

### Example 11

### PKG2 Induces DUSP10 (MKP5) Expression and Inhibits JNK-P

### Materials and Methods

The relative expression of DUSP10 was measured by RT-PCR in wild type and knockout mice (Prkg2+/+ and Prkg2−/−), LS174T and HT29 colon cancer cells with or without PKG2 expression, and in colon explants treated with 8BrcGMP. Mucosa from different parts of the mouse digestive tract were analyzed by immunoblotting for JNKP, total JNK, DUSP10 and PKG2. Immunoblotting of ileum and proximal colon explants with or without 8Br-cGMP treatment to activate PKG2 was also performed.

### Results

To gain some insight into potential molecular mechanisms underlying the PGK2 knockout phenotype, a gene-array study was performed with intestinal mucosa of the PKG2 knockout. Two genes that stood out were DUSP8 and DUSP10, which are inhibitors of cJun N-terminal kinase (JNK) phosphorylation/activation. JNK is involved in both the reactivity of colonocytes and their apoptosis. Upregulation of DUSP genes was investigated as a potential mechanism for PKG2 function in the colon epithelium. DUSP8/10 expression by PKG2 was confirmed by RT-PCR in colons from WT vs KO mice, in colon explants treated with cGMP, and in some colon cancer cell lines. Expression of PKG2 along the GI tract was found to be inversely associated with basal activated JNK-P, and levels decrease in colon explants treated with cGMP.

### Example 12

### Vardenafil Treatment is Protective in a Murine Colitis Model

### Materials and Methods

Vardenafil was administered beginning 2 days prior to introducing 5% dextran sulphate sodium (DSS) into the drinking water of CD1 mice. Control animals were injected with PBS vehicle. After 5 days the DSS was replaced with water. Incidence and severity of diarrhea and body weight were recorded daily and combined in an index of disease severity. Error bars indicate standard deviation and asterisks show statistical difference between Vardenafil treated and Controls at each day (p<0.05, n=4 for each group).

### Results

The data described above indicates that the protective effect of PKG2 can be enhanced in the colon using systemic vardenafil administration. The ability to protect the luminal surface of the colon from toxins or inflammatory stimuli by (pre) treating the animals with Vardenafil was also tested.

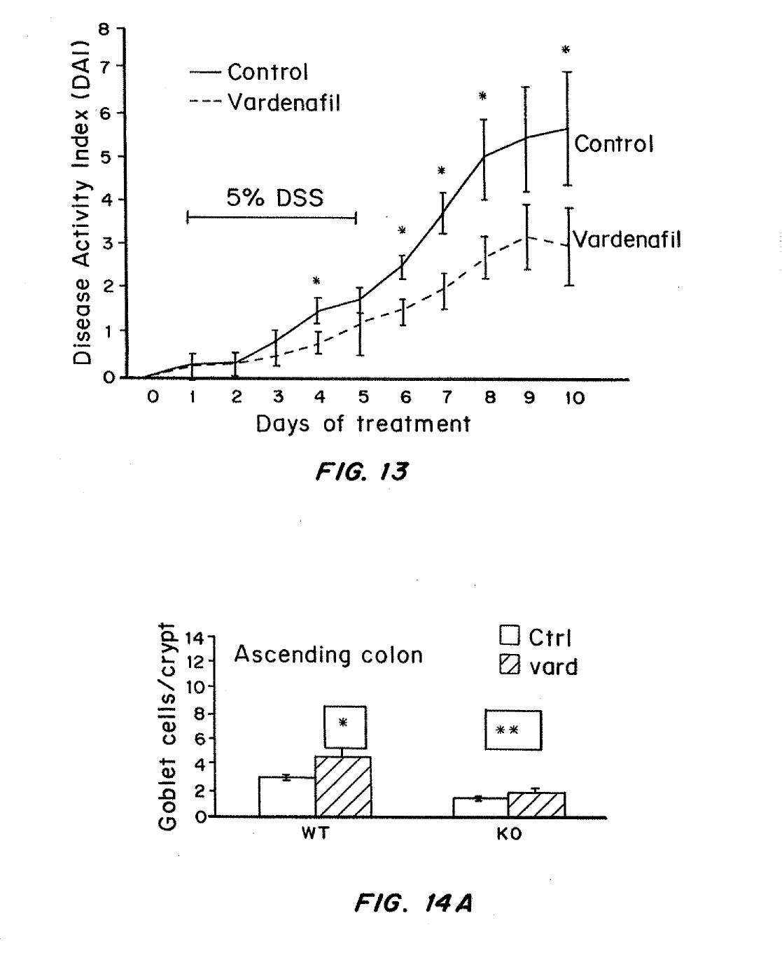

The best characterized mouse model of IBD involves administration of dextran sulfate-sodium (DSS) in the drinking water, and this is toxic specifically to luminal epithelial cells of the distal colon. Since this preferentially affects distal colon, it is a model for human ulcerative colitis, and maybe not the best model for our studies because the effects of Vardenafil were less pronounced in the distal colon compared to the proximal colon. Despite these issues, the effect of Vardenafil was tested on animals treated with maximal concentrations of DSS. Animals were pretreated with Vardenafil (IP/B.I.D.) for 2 days and treatment was maintained throughout the disease model (10 days). FIG. 13 shows a significant protective effect of Vardenafil. The 5% DSS used was on the high end and resulted in death in half the untreated animals and but only 25% of the treated animals (not shown, n=4 in both groups). This results is supportive of the use of FDA approved PDE5 inhibitors for the therapeutic benefit of patients with inflammatory bowel disease (IBD).

### Example 13

### PKG2 Mediates Both the Goblet Cell Differentiation and Growth/Death Effects of Vardenafil Treatment

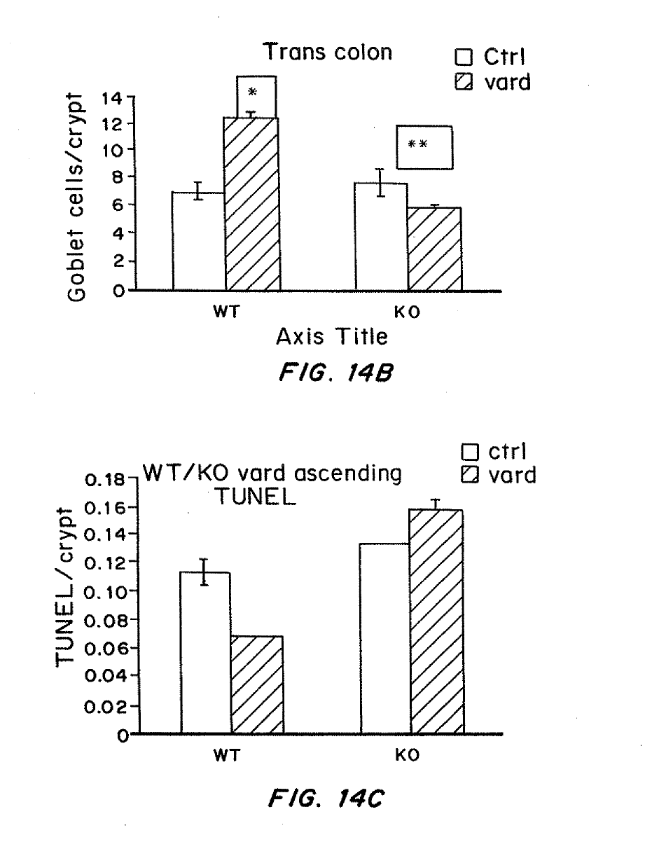

FIGS. 14A-14C show quantification of mature goblet cells in the ascending (A) and transverse colon (B) of wild type and PKG2 knockout animals. The animals were either treated with Vardenafil (5 mg/kg, IP, b.i.d.) for 7 days or injected with vehicle. The increase in goblet cells observed in the wild type animals was not observed in the PKG2-deficient animals, which indicates the involvement of PKG2 in this process. The reduction in apoptosis in the wild type animals in response to vardenafil treatment was not observed in the PKG2 knockouts (C). These results support the idea that PKG2 mediates both the goblet cell differentiation and growth/death effects of vardenafil treatment.

### Example 14

### Vardenafil Treatment Reduces Reduction-Oxidation (Redox) Stress Levels in the Colon Mucosa

### Materials and Methods

Antioxidant Expression

Explants of proximal and distal colon from mice were treated for 3-4 hours with 100 uM 8Br-cGMP and then the mucosa was harvested for analysis of antioxidant gene and protein expression by RT-PCR and Western blotting. Blots were reprobed for b-actin expression as a loading control.

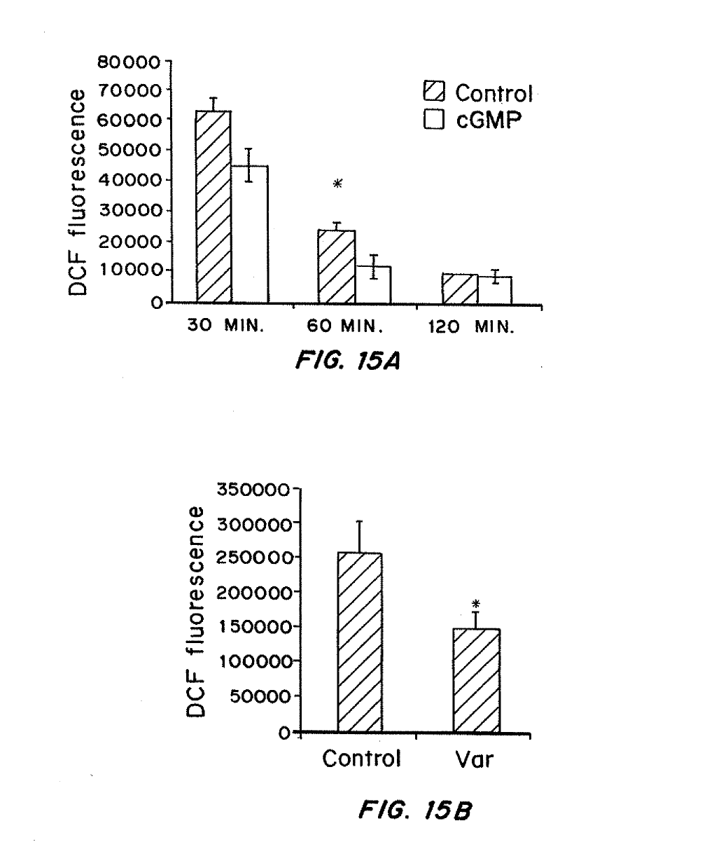

CD1 mice were treated with 30 mg of vardenafil (IP) or with vehicle (100 μl olive oil). After 4 h the animals were sacrificed and the colon mucosa was harvested for analysis of redox stress using DCF fluorescence. Error bars indicate standard deviation (n=3), but p>0.05 by t-test (FIG. 15B)

Dichlorofluorescein (DCF) Assay

Colons were removed from sacrificed animals and incubated with 20 μM DCF for 30 minutes, then homogenized and the fluorescence measured (at FITC wavelengths; 485/535 nm). Specimens were incubated directly (30 min time point), or incubated for 30 min or 90 min under standard tissue culture conditions before adding DCF for the final 30 min (60 and 120 min time points).

### Results

PKG1 has been found to activate Foxo4 in colon cancer cells (Kwon, et al., Oncogene, 29(23): 3423-3434 (2010)). Activation of Foxo4 was found to be the central mechanism underlying the inhibition of b-catenin/TCF-dependent proliferative signaling in these cells. In addition to attenuating proliferation, the activation of Foxo4 can upregulate antioxidant genes. To determine whether activation of endogenous PKG could affect the expression of antioxidant genes in the colon, explants of proximal and distal colon from mice were treated for 3-4 hours with 100 uM 8Br-cGMP and then the mucosa was harvested for analysis of antioxidant genes expression by RT-PCR or Western blotting. HPRT and b-actin were used as loading controls.

This approach demonstrated a broad increase in antioxidant genes that ostensibly went beyond just Foxo targets, indicating that (1) activation of antioxidant gene expression by multiple pathways might be a fundamental role for PKG in the colon, or (2) that PKG activation could itself induce redox stress which increased antioxidant gene expression. To test the possibility that PKG activation could itself induce redox stress, colon mucosa was tested at different time points following treatment of colon explants with 100 uM 8Br-cGMP (FIG. 15A). These experiments show that cGMP treatment did not increase redox stress, but instead decreased it. This finding demonstrates that the increased antioxidant genes was not due to redox stress induced by cGMP/PKG, but instead indicates that the increase in gene expression is likely to be mediated by specific signaling pathways and that it could effectively reduce redox stress. To determine whether elevating cGMP in the colon could reduce redox stress in vivo, mice were treated with 5 mg/kg vardenafil (IP) for 4 hrs. The proximal colon was removed and redox stress in the mucosa was measured using DCF fluorescence as a readout (FIG. 15B). This study demonstrated that vardenafil treatment (var) could indeed affect redox stress levels in the colon mucosa.

### Example 15

### PKG2 Plays a Central Role in Regulating JNK Signaling

### Methods

Immunohistochemistry of tissue samples from Vardenafil treated and control mice was performed. WT mice were treated without (control) or with vardenafil as described above for 7 days and colons were harvested and stained for goblet cells (AB&PAS), proliferative cells (Ki-67) and apoptotic cells (TUNEL) in the crypts.

### Results

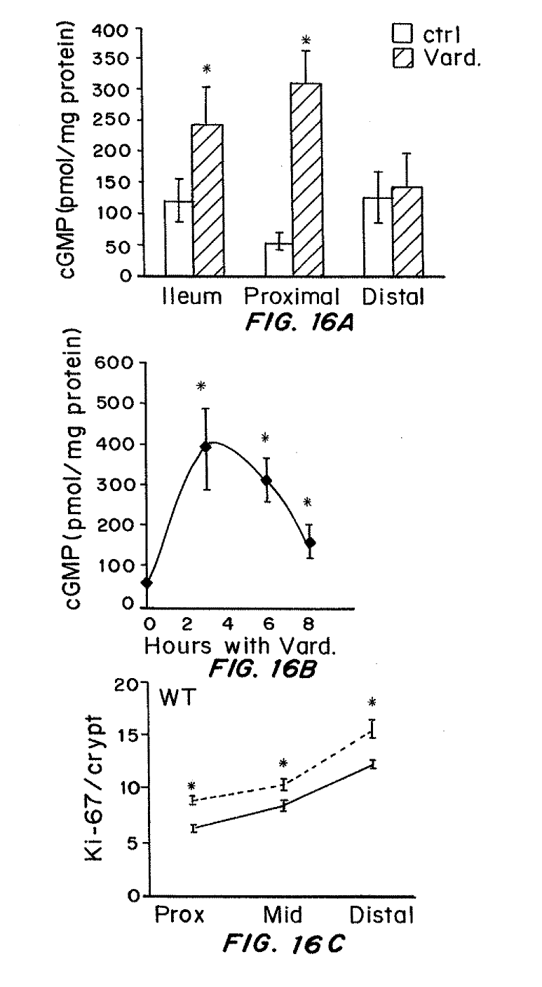

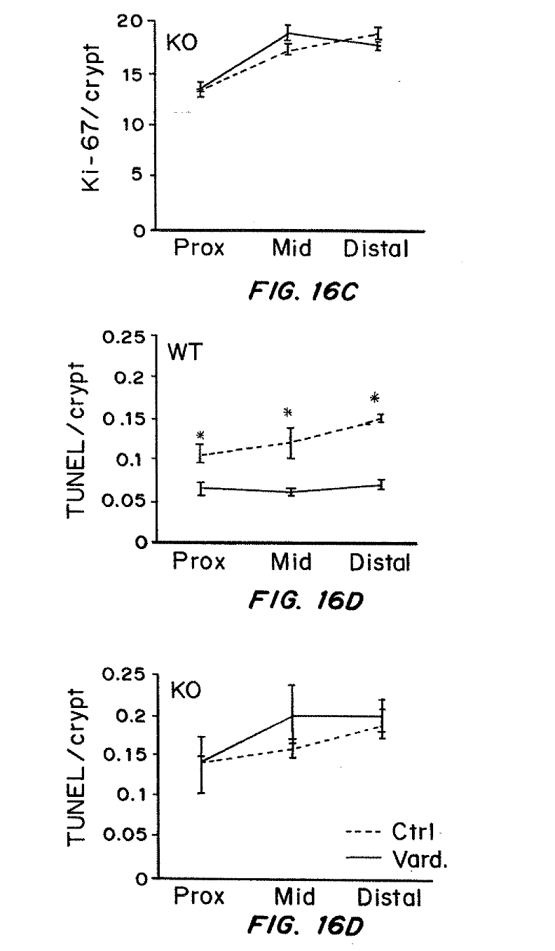

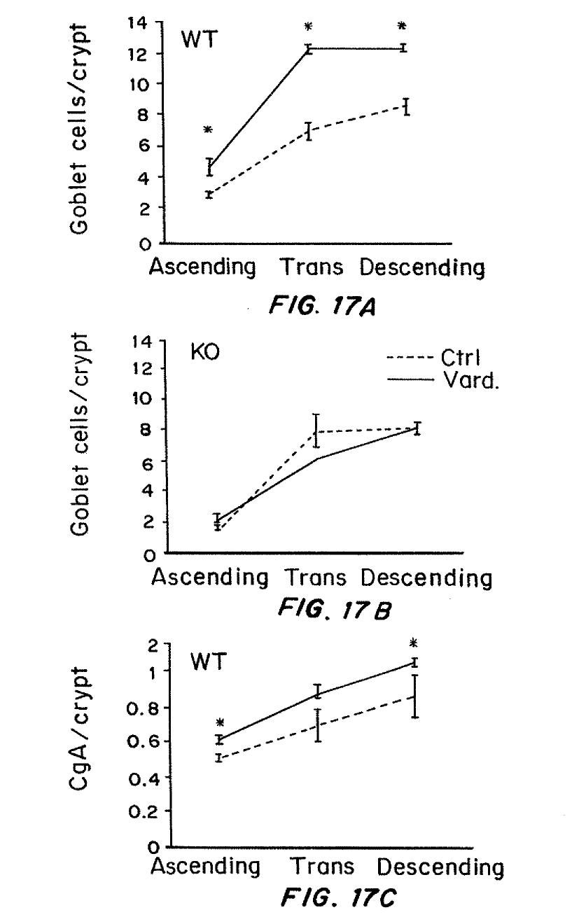

Increasing cGMP levels has emerged as a potentially important suppressor of tumorigenesis in the colon, but the downstream signaling is poorly defined and a viable therapeutic strategy remains to be established. PKG2 was identified as playing a role in homeostasis in the colon mucosa by analyzing Prkg2^{-/-} mice, which showed crypt hyperplasia and reduced differentiated secretory cells relative to Prkg2^{+/+} siblings (AJP (Gastro), 303(2): G209-19). The significance of this pathway was tested in the present study using the PDE5 inhibitor Vardenafil (Levitra™), which rapidly increased cGMP in the proximal colon epithelium, and to a lesser extent, in the terminal ileum and distal colon (FIG. 16a,b). Mice treated with Vardenafil (i.p.) for several days had reduced proliferation and apoptosis in colonic crypts and a dramatic increase in mature goblet cells (FIG. 16c,d; FIG. 17). Consistent with the importance of PKG2 in this process, Vardenafil did not affect homeostasis in Prkg2^{-/-} animals.

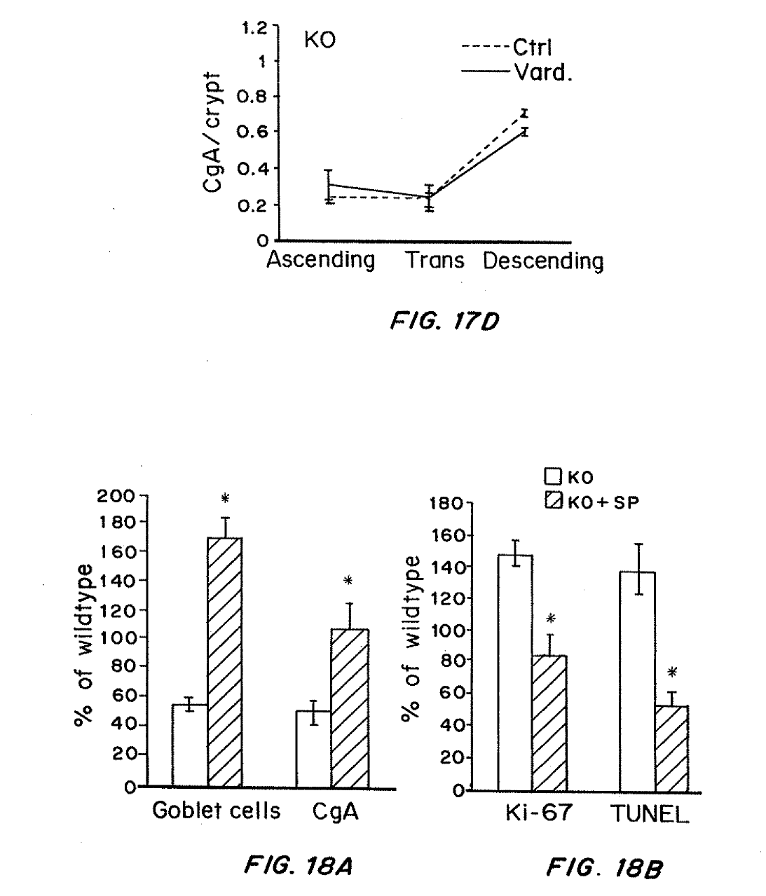

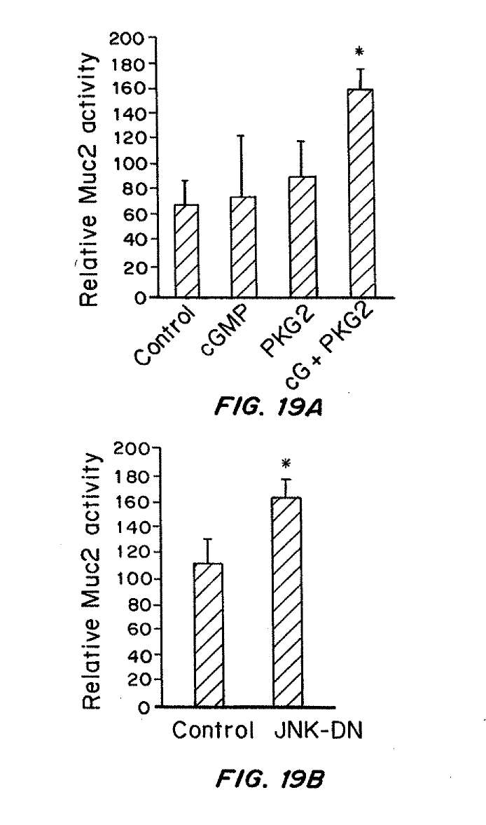

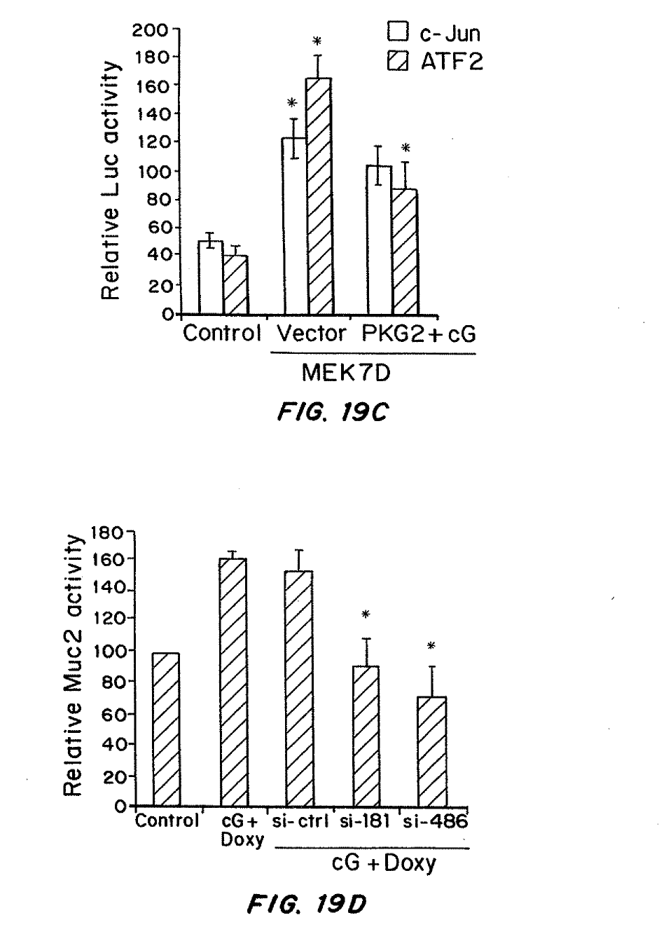

To better understand the mechanism, gene expression in the colon mucosa from Prkg2^{-/-} and Prkg2^{+/+} was explored using micro-arrays. This work identified dual-specificity phosphatase 10 (DUSP10) as a potentially important effector of PKG2. DUSP10 suppresses cJun N-terminal kinase (JNK) by dephosphorylating the active form of the enzyme. DUSP10 expression was significantly reduced in the colon mucosa of Prkg2^{-/-} compared to Prkg2^{+/+} mice, and activation of PKG in the colon mucosa in vitro, increased DUSP10 expression and reduced phospho-JNK levels. Notably, treatment of Prkg2^{+/+} with the JNK inhibitor SP600125 restored proliferation and differentiation to near wild type levels, indicating that regulation of JNK is a central function of PKG2 in the colon (FIG. 18a, b). LS174T colon cancer cells retain the ability of PKG2 to upregulate Muc2 and DUSP10 expression, and inhibit JNK activity (FIG. 19a, b, c). Silencing of DUSP10 using siRNA blocked Muc2 expression and JNK inhibition by PKG2 (FIG. 19d).

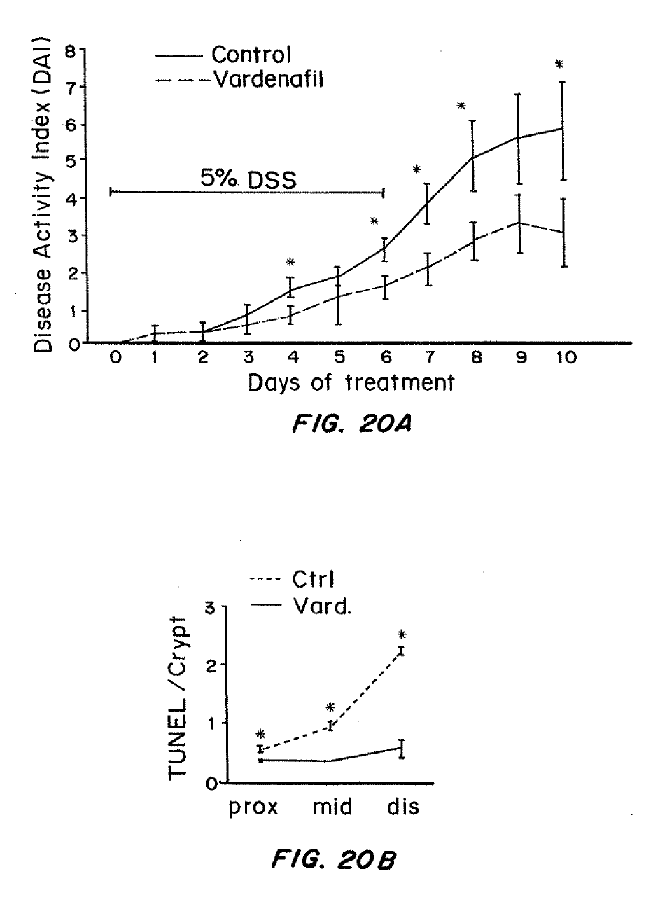

Treatment of wild type mice with Vardenafil increased DUSP10 levels in the colon mucosa, and this inhibited dextran sulfate sodium (DSS) induced activation of both JNK, apoptosis and severity of tissue destruction as measured by gastrointestinal symptoms (FIG. 20). Immunohistochemistry of descending colons of DSS mice treated with or without vardenafil stained with DAPI and JNK-phospho T183/Y185 showed that Vardenafil reduced or inihibited JNK activity.

Systemic administration of Vardenafil (PDE-5 inhibitor, Levitra) can effectively increase cGMP levels in the colon mucosa, and protects against epithelial damage. In the colon epithelium, Vardenafil suppresses proliferation and apoptosis, while elevating the number of secretory lineage cells in a PKG2-dependent manner. A central component of PKG2 signaling is the activation of DUSP10 expression, which increases survivability by suppressing JNK in the colon epithelium. In summary, the results demonstrate a central role of PKG2 regulating JNK signaling in the colon epithelium, and that this pathway is activated by treatment with PDE5 inhibitors. The barrier-protective effect of JNK blockade indicates the use of these agents as part of a therapeutic strategy for treating colitis and colon cancer prevention.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A method of treating an intestinal or bowel disorder comprising administering to a subject an effective amount of a composition that increases cGMP level in the intestinal mucosa or increases the activity of a PKG in intestinal mucosa to reduce one or more symptoms of the intestinal bowel disorder.

2. The method of claim 1 wherein the composition comprises one or more phosphodiesterase 5 inhibitors.

3. The method of claim 2 wherein the phosphodiesterase 5 inhibitor is vardenafil.

4. The method of claim 1 wherein the intestinal bowel disorder is an inflammatory bowel disease.

5. The method of claim 4 wherein the inflammatory bowel disease is selected from the group consisting of Crohn's disease, ulcerative colitis, necrotizing enterocolitis, collagenous colitis, lymphocytic colitis, ischaemic colitis, diversion colitis, Behçet's disease, and indeterminate colitis. Symptoms of inflammatory disorders include abdominal pain, vomiting, diarrhea, rectal bleeding, severe internal cramps/muscle spasms in the region of the pelvis, weight loss, arthritis, pyoderma gangrenosum, and primary sclerosing cholangitis.

6. The method of claim 4 wherein one or more of the symptoms is selected from the group consisting of abdominal pain, vomiting, diarrhea, rectal bleeding, severe internal cramps/muscle spasms in the region of the pelvis, weight loss, arthritis, pyoderma gangrenosum, and primary sclerosing cholangitis.

7. The method of claim 1 wherein the inflammatory bowel disorder is irritable bowel syndrome (IBS).

8. The method of claim 7 wherein one or more of the symptoms is selected from the group consisting of abdominal pain or discomfort, frequent diarrhea or constipation, a change in bowel habits, urgency for bowel movements, a feeling of incomplete evacuation (tenesmus), bloating or abdominal distention, gastroesophageal reflux, symptoms relating to the genitourinary system, chronic fatigue syndrome, fibromyalgia, headache, and backache.

9. A method of improving intestinal luminal integrity comprising administering to a subject an effective amount of a composition that increases cellular level of cGMP in the intestinal mucosa or increases the activity of a PKG in the intestinal mucosa to increase or improve one or more physiological parameters of intestinal luminal integrity.

10. The method claim 9 wherein reactivity of luminal epithelial cells in response to inflammatory stimuli is reduced.

11. The method of claim 9 wherein the function of the integrity of the luminal surface is strengthened or enhanced.

12. The method of claim 9 wherein the one or more physiological parameters of intestinal luminal integrity are selected from the group consisting of reduced proliferation at the base of the intestinal crypts, reduced apoptosis at the intestinal luminal surface, induction of downstream effectors of PKG mediated signaling in the intestinal mucosa, increased DUSP8/10 (MKP5) expression or activation in the intestinal mucosa, decreased JNK expression or activation in the intestinal mucosa, decreased Sox9 protein levels in the intestinal mucosa, decreased Sox9 inhibition Sox9 repressive activity in the intestinal mucosa, increased activity of an antioxidant in the intestinal mucosa, increased expression of one or more genes encoding an antioxidant including, but not limited to, MnSOD, PRDX3, Cat, GSTa3, GSR, GPX1, or GPX2, in the intestinal mucosa, or reduce redox stress in the intestinal mucosa compared to a control.

13. The method of claim 9, wherein the composition comprises one or more phosphodiesterase 5 inhibitors.

14. The method of claim 13, wherein the phosphodiesterase 5 inhibitor is vardenafil.

16. The method of claim 13 wherein the phosphodiesterase 5 inhibitor is selected from the group consisting of avanafil, lodenafil, mirodenafil, sildenafil citrate, tadalafil, udenafil, zaprinast, and icariin.

17. The method of claim 9, wherein the composition increases the activity of PKG2 in the intestinal mucosa.

18. The method of claim 9, wherein the composition is targeted to the intestinal mucosa.

19. A method of treating an inflammatory bowel disease comprising administering to a subject in need thereof an effective amount of a composition to reduce or alleviate one or more symptoms of an inflammatory bowel disease, wherein the composition comprises a compound defined by Formula I 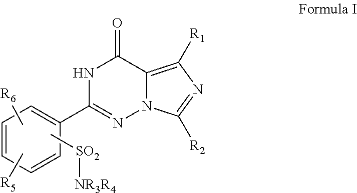
wherein
R₁, R₂, R₅, and R₆ are, independently, hydrogen, a straight-chain or branched alkenyl or alkoxy group having up to 8 carbon atoms, or a straight-chain or branched alkyl chain having up to 10 carbon atoms which is optionally interrupted by an oxygen atom, and which optionally contains one or more substituents selected from trifluoromethyl, trifluoromethoxy, hydroxyl, halogen, carboxyl, amino, nitro, benzyloxycarbonyl, straight-chain or branched alkoxycarbonyl having up to 6 carbon atoms; and
R₃ and R₄, together with the nitrogen atom to which they are attached, form a 5- to 7-membered unsaturated or saturated or partially unsaturated heterocycle, optionally containing up to 3 heteroatoms selected from S, N and O or a radical of the formula —NR₃₇—, in which R₃₇ represents hydrogen, hydroxyl, formyl, trifluoromethyl, a straight-chain or branched acyl group, alkoxy group, or alkoxycarbonyl group having in each case between 1 and 6 carbon atoms, or a straight-chain or branched alkyl group having between 1 and 6 carbon atoms, optionally substituted with one or more substituents selected from hydroxyl, trifluoromethyl, carboxyl, amino, nitro, or a straight-chain or branched alkoxy or alkoxycarbonyl group having between 1 and 6 carbon atoms;
or a pharmaceutically acceptable analog, salt, solvate, N-oxide, or isomeric form thereof.
